(19) United States
(12) Patent Application Publication
(10) Pub. No.: US 2016/0272937 A1
(43) Pub. Date: Sep. 22, 2016

(54) METHODS OF GENERATING INTERMEDIATE MESODERM CELLS FROM HUMAN PLURIPOTENT STEM CELLS

(71) Applicant: THE BRIGHAM AND WOMEN'S HOSPITAL, INC., Boston, MA (US)

(72) Inventors: Albert LAM, Brookline, MA (US); Benjamin FREEDMAN, Boston, MA (US); Ryuji MORIZANE, Brookline, MA (US); Joseph BONVENTRE, Wayland, MA (US)

(73) Assignee: THE BRIGHAM AND WOMEN'S HOSPITAL, INC., Boston, MA (US)

(21) Appl. No.: 15/030,315

(22) PCT Filed: Apr. 14, 2014
(86) PCT No.: PCT/US2014/034031

§ 371 Date: Apr. 18, 2016

Related U.S. Application Data
(60) Provisional application No. 61/891,546, filed on Oct. 16, 2013.

(51) Int. Cl. C12N 5/0735; A61K 35/54

(52) U.S. Cl. CPC C12N 5/0611; A61K 35/54; C12N 2501/115; C12N 2501/385; C12N 2501/16; C12N 2501/119; C12N 2506/02; C12N 2500/90; C12N 2506/45; C12N 2501/727

(57) ABSTRACT

Described herein are methods related to generating intermediate mesoderm (IM) cells, including using sequential treatment of small molecules and growth factors, and composition produced by the described methods. Using small molecules such as CHIR99021 in combination with FGF2 and RA, efficient differentiation of human pluripotent stem cells (hPSCs) into intermediate mesoderm, such as PAX2+LHX1+ cells, is achieved. The method is extensible different hPSC cell lines and does not require flow sorting. Importantly, resulting PAX2+LHX1+ cells, are capable of WT1 expression and addition of FGF9 and activin, PAX2+LHX1+ cells specifically differentiates cells into SIX2, SALL1, and WT1 expressing cells representative of cap mesenchyme nephron progenitor cells. The described methods and compositions facilitate and improve the directed differentiation of hPSCs into cells of the kidney lineage for the purposes of bioengineering kidney tissue and iPS cell disease modeling.

### FIELD OF INVENTION

Descried herein are methods and compositions related to production of intermediate mesoderm cells from pluripotent stem cells. The techniques described herein find use in regenerative medicine applications.

### BACKGROUND

Chronic kidney disease (“CKD”) is a significant global public health problem and is the leading risk factor for cardiovascular disease. In spite of advances in the quality of dialysis therapy, patients with CKD experience significant morbidity and mortality and reduced quality of life. For selected patients, kidney transplantation is an alternative renal replacement therapy to dialysis; however, this option is limited by the shortage of compatible organs and requires the use of lifelong immunosuppressive medication to prevent graft rejection. For these reasons, research in regenerative medicine, with the ultimate aim of generating functional replacement kidney tissue or even a whole kidney from a patient's own tissue, offers the potential for new therapeutic strategies to treat CKD and end stage renal disease (“ESRD”). Human pluripotent stem cells (“hPSCs”) have the revolutionary potential to generate functional cells and tissues for purposes of regenerative medicine and disease modeling. Both human embryonic stem cells (“hESCs”) and human induced pluripotent stem cells (“hiPSCs”), which are each members of the broader category of human pluripotent stem cells (“hPSCs”), possess the ability to self-renew and to differentiate into cells from all three germ layers of the embryo. This plasticity of hPSCs makes them an ideal resource for generating cells of the kidney lineage.

While other organs such as the heart, liver, pancreas, and central nervous system have benefited from more established differentiation protocols for deriving their functional cell types from hPSCs, considerably fewer methods have been developed to effect kidney differentiation. This may be partly explained by the complex architecture of the kidney and its functional units, nephrons, which are comprised of highly specialized epithelial cell types, such as glomerular podocytes, proximal tubular epithelial cells, cells of the thick and thin limbs of the loop of Henle, distal convoluted tubule and collecting duct cells. Thus, there is a great need in the art to establish a system capable of differentiating hPSCs into these nephrontic cell populations, further including establishment of protocols for generating nephron progenitor cell populations. Nephron cell progenitors, such as intermediate mesoderm (“IM”) and the metanephric mesenchyme, which can offer a common starting platform for derivation of specific kidney lineage cells.

Described herein is a simple, efficient, and highly reproducible system to induce IM differentiation in hESCs and hiPSCs under chemically defined, monolayer culture conditions. Chemical induction with the potent small molecule inhibitor of GSK3β, CHIR99021 (“CHIR”), robustly and rapidly differentiates hPSCs to a multipotent mesendoderm stage in a manner that recapitulates mesendoderm formation in the developing embryo. Further, hPSCs treated with CHIR can preferentially differentiate into lateral plate mesoderm, but with precisely timed addition of specific growth factors, this default fate can be diverted into definitive endoderm or other types of mesoderm. Importantly, treatment with CHIR, and the combination of FGF2 and retinoic acid (“RA”) effectively generates IM cells co-expressing PAX2 and LHX1 within 3 days of differentiation. This is the most efficient method to generate PAX2+LHX1+IM cells and the first demonstration of the role of FGF signaling as a potent inducer of IM-specific PAX2 expression in differentiating hPSCs. These PAX2+LHX1+ cells form tubular structures which express apical cilia and markers of proximal tubular epithelial cells and integrate into mouse embryonic kidney explant cultures, demonstrating their capacity to give rise to IM derivatives. In addition, PAX2+LHX1+ cells can also be specifically differentiated into cells expressing SIX2, SALL1, and WT1, markers of the multipotent nephron progenitor cells of the cap mesenchyme (CM), further demonstrating their capacity to give rise to IM derivatives.

### BRIEF DESCRIPTION OF FIGURES

This patent or application file contains at least one drawing executed in color. Copies of this patent or patent application publication with color drawings will be provided by the Office upon request and payment of the necessary fee.

FIG. 1. CHIR99021 efficiently induces human pluripotent stem cells to differentiate into mesendoderm. (A) Schematic diagram of differentiation of human PSCs into mesendoderm using CHIR. (B) hPSCs treated with DMSO (vehicle), Wnt3a 500 ng/mL, Wnt3a 500 ng/mL+activin 50 ng/mL, or CHIR 5 μM were immunostained for BRACHYURY after 24 hours of differentiation. (C) Quantification of cells with positive immunofluorescence staining for BRACHYURY after treatment with vehicle, Wnt3a at increasing doses, Wnt3a at increasing doses with activin, and CHIR for 24 hours. Data represent means±s.e.m. (n=3). (D) Time course of gene expression in hPSCs treated with CHIR. Quantitative RT-PCR of genes expressed in mesendoderm and in pluripotent stem cells. Data represent means±s.e.m. (n>3). (E) Immunofluorescence staining for BRACHYURY and MIXL1 in CHIR-treated hPSCs over the first 48 hours of differentiation. (F) Immunofluorescence staining for BRACHYURY and MIXL1 after 24 hours of differentiation in hESC and hiPSC lines treated with CHIR. (G) Phase contrast images of CHIR-treated human PSCs over 36 hours showing epithelial to mesenchymal transition. (H) Immunofluorescence staining for E-cadherin and N-cadherin in CHIR-treated human PSCs. Scale bars, 100 μm. BRACHY, BRACHYURY; CHIR, CHIR99021; ME, mesendoderm; PSC, pluripotent stem cell.

FIG. 2. Timed addition of exogenous factors modulates cell fate of CHIR-induced hPSCs. (A) Schematic diagram depicting time course of differentiation. (B) Time course of gene expression in human PSCs treated with CHIR for 24 hours, CHIR for 48 hours, or DMSO (vehicle). Data shown represent means±s.e.m. (n=3). Representative immunostaining (C) and quantification (D) of markers of pluripotency, mesoderm, definitive endoderm, and ectoderm in human PSCs treated with DMSO (vehicle), CHIR for 24 hours, or CHIR for 48 hours, day 4. Data shown in graph represent means±s.e.m. (n>5). (E) Expression of BMP-4 by quantitative RT-PCR in hPSCs treated with CHIR for 24 hours, CHIR for 48 hours, or DMSO. Data shown represent means±s.e.m. (n=3). (F) Representative immunostaining for SOX17 and FOXA2 in hESCs and hiPSCs treated with CHIR for 24 hours followed by activin A 100 ng/mL for 3 days. Numbers represent the mean percentage±s.e.m. of SOX17+ cells from at least 2 independent experiments. (G) Cells at the definitive endoderm stage were differentiated using a three step protocol into hormone-expressing pancreatic endocrine cells producing insulin, pro-insulin Cpeptide, and somatostatin. Number represents the mean percentage±s.e.m. of insulin+Cpeptide+ cells from at least 2 independent experiments. (H) Schematic diagram of directed differentiation of hPSCs into intermediate mesoderm. (I) Quantification of cells with positive immunostaining for PAX2. Data shown in graph represent means±s.e.m. (n=2). (J) Immunostaining for PAX2 in hPSCs treated with or without CHIR for 24 hours followed by FGF2 100 ng/mL for 3 days, day 4. Scale bars, 100 μm.

FIG. 3. FGF2 and retinoic acid induce PAX2+LHX1+ intermediate mesoderm cells. (A) Immunostaining for PAX2 and LHX1 in hPSCs cells treated with CHIR for 24 hours followed by FGF2 or FGF2+ retinoic acid (RA) for 3 days, day 4. (B) Quantification of PAX2+ cells on days 2-7 of differentiation in hPSCs treated with CHIR for 24, 36, or 48 hours followed by FGF2+RA. Data shown represent means±s.e.m. (n>4) (C) Immunostaining for PAX2 and LHX1 and (D) quantification of PAX2+ cells in three hESC lines and two hiPSC lines treated with CHIR for hours followed by FGF2+RA (ChFR), day 3. Data shown represent means±s.e.m. (n=3) (E) Quantification of PAX2+ and LHX1+ cells in hPSCs treated with CHIR for 36 hours followed by FGF2+RA or FGF2+RA+BMP-7, day 3. Data shown represent means±s.e.m. (n=3). (F) Quantification of PAX2+ and LHX1+ cells by flow cytometry. (G) Quantitative RT-PCR of intermediate mesoderm genes in hPSCs treated with ChFR. Data shown represent means±s.e.m. (n=3). (H) Immunostaining for LHX1 and WT1 in hPSCs treated with ChFR. (I) Quantitative RT-PCR of non-IM genes in hPSCs treated with ChFR. Data shown represents means±s.e.m. (n=2). Scale bar, 100 μm.

FIG. 4. PAX2+LHX1+ intermediate mesoderm cells form polar tubular structures expressing polycystin-2+ cilia and kidney proximal tubular markers. (A) Immunostaining time course from days 3 to 9 for PAX2, kidney-specific protein (KSP), and Lotus tetragonolobus lectin (LTL) in hPSCs treated with ChFR for 3 days, then cultured in media without additional growth factors for an additional 6 days. Scale bar, 50 μm. (B) Immunostaining for KSP and LTL in tubular structures formed by PAX2+LHX1+IM cells, day 9. Inset shows tubular structure at higher magnification. Scale bar, 50 μm. (C) Brightfield stereomicroscope imaging of tubular structures, day 9. Scale bar, 200 μm. (D) Quantification of tubular structures formed from PAX2+LHX1+IM cells, day 9. Data shown represent means±s.e.m. (n=4 for each hES and hiPS cell line). (E) Immunostaining for KSP, LTL, and laminin in tubular structures derived from two hESC and one iPSC lines, day 9. Scale bar, 50 μm. (F) Immunostaining for LTL and N-cadherin in tubular structures derived from one hESC and one iPSC line, day 9. Scale bar, 50 mm. (G) Immunostaining for acetylated a-tubulin and polycystin-2 in tubular structures, day 9. Inset shows higher magnification of cilia localized to the apical surface. Scale bar, 50 mm. (H) Quantitative RT-PCR of genes associated with kidney development and mature kidney epithelial cells. Data shown represent mean6SEM(n=2). (I) Whole-mount immunohistochemistry for anti-human nuclear antigen (HNA) and laminin in chimeric kidney explant cultures. Dissociated hPSC-derived IM cells on day 3 (n=10) and day 9 (n=3) of differentiation were mixed with dissociated E12.5 mouse embryonic kidneys in single cell suspension, reaggregated by centrifugation, and cultured for 3 days in kidney explant culture. Arrowhead, laminin-bounded structures containing human and mouse cells. Scale bar, 50 mm. AQP1, aquaporin-1; AQP2, aquaporin-2; DAPI, 49,6-diamidino-2-phenylindole; HNA, human nuclear antigen; UMOD, uromodulin.

FIG. 5. Efficient differentiation of definitive endoderm and endodermal derivatives from CHIR-induced mesodermal cells. (A) Quantification of SOX17+ cells at day 4 of differentiation. Undifferentiated hPSCs (Day 0) serve as the negative control. (B) Quantification of SOX17+ cells at day 4 of differentiation of hPSCs treated with activin A at either 24 or 48 hours after treatment with CHIR. (C) Quantitative RT-PCR of definitive endoderm genes in hPSCs treated with CHIR or CHIR+ activin A. Gene expression normalized to Day 0. (D) hPSCs differentiated into definitive endoderm with CHIR+ activin A were further differentiated into hepatocytes. Immunostaining for stage-specific markers at each step of differentiationis shown. (E) Cells at the definitive endoderm stage were differentiated with SB431542 and Noggin for 3 days into anterior foregut endoderm. (F) Cells at the definitive endoderm stage were further differentiated with FGF4 and CHIR for 4 days into hindgut endoderm expressing CDX2 without expression of foregut differentiation markers PDX1 or albumin. A100, activin A, 100 ng/mL; ALB, albumin; C-PEP, pro-insulin C-peptide; INS, insulin; SS, somatostatin. Data represent means±s.e.m. (n=3). Scale bar, 100 μm.

FIG. 6. PAX2+LHX1+ intermediate mesoderm cells can be differentiated further into SIX2+WT1+ of the metanephric mesenchyme. (A) Schematic diagram showing the stages of differentiation from hPSCs into mesendoderm, intermediate mesoderm, and metanephric mesenchyme. Growth factors used in the protocol are shown about the arrows. (B) Immunocytochemistry for PAX2, LHX1, WT1, and SIX2 in hPSCs treated with CHIR for 36 hours, then FGF2 100 ng/mL+RA 1 μm for 42 hours, then FGF9 100 ng/ml+Activin A 10 ng/mL, Days 3, 6, and 9. SIX2 and WT1 co-expression is observed as early as day 6 of differentiation. (C) Quantitative RT-PCR of genes expressed in the intermediate mesoderm and metanephric mesenchyme in hPSCs treated with CHIR for 36 hours, FGF2+RA for 42 hours, then FGF9 and activin A. SIX2 and WT1 expression are highly upregulated on Day 6 compared to Day 0 and Day 3.

FIG. 7. FGF-9 and activin differentiate PAX2+LHX1+ cells into cells expressing markers of CM. (A) Whole-mount immunohistochemistry for anti-human nuclear antigen (HNA) and SIX2 in chimeric kidney explant cultures. Dissociated hESC-derived IM cells on day 9 (n=3) of differentiation were mixed with dissociated E12.5 mouse embryonic kidneys in single cell suspension, reaggregated by centrifugation, and cultured for 5 days in kidney explant culture. Arrowhead, HNA+ cells present within clusters of mouse Six2+ cells. Scale bar, 50 mm. (B) Diagram showing the stepwise differentiation of hESCs into metanephric CM. (C) Immunostaining for SIX2 in hESCs treated with FGF2+RA (ChFR) for 3 days then either 100 ng/ml FGF-9, 10 ng/ml activin, or vehicle for 3 days, day 6. Scale bar, 100 mm. (D) Immunostaining for SIX2 in hESCs plated at different densities and treated with ChFR for 3 days then 100 ng/ml FGF-9+10 ng/ml activin for 3 days, day 6. Scale bar, 100 mm. (E) Immunostaining for SIX2, SALL1, and WT1 in hESCs treated with ChFR for 3 days then 100 ng/ml FGF-9+10 ng/ml activin for 3 days, day 6. Dashed line encompasses the population of cells that stain positive for WT1.

### SUMMARY OF THE INVENTION

Described herein is a method for generating a mesoderm cell, including providing a quantity of human pluripotent stem cells (“hPSCs”), and culturing the hPSCs in a serum-free media including at least one induction molecule, wherein the at least one induction molecule is capable of generating at least one mesoderm cell. In some embodiments, the human pluripotent stem cells are human embryonic stem cells (“hESCs”). In some embodiments, the human pluripotent stem cells are human induced pluripotent stem cells (“hiPSCs”). In other embodiments, the at least mesoderm cell is an intermediate mesoderm cell. In other embodiments, the intermediate mesoderm cell expresses paired box-2 (“PAX2”), LIM homeobox-1 (“LHX”), and/or Wilms tumor-1 (“WT1”). In other embodiments, the at least one induction molecule is a Glycogen synthase kinase-3 beta (“GSK3β”) inhibitor. In other embodiments, the GSK3β inhibitor is CHIR99021. In other embodiments, the hPSCs are cultured in a serum-free media comprising at least one induction molecule for about 12, 24, 36, or 48 hours. In certain embodiments, the method includes further culturing of the at least one mesoderm cell in the presence of at least one growth factor. In other embodiments, the at least one growth factor includes fibroblast growth factor-2 (“FGF2”) and/or retinoic acid (“RA”). In other embodiments, the method includes further culturing of the at least one mesoderm cell in the presence of FGF2 and/or RA is for about 36, 48, 60, or 72 hours. In other embodiments, the method includes further culturing in the presence of fibroblast growth factor-9 (“FGF9”) and/or activin A.

Also described herein is a composition of at least one mesoderm cell generated by a method for generating a mesoderm cell, including providing a quantity of human pluripotent stem cells (“hPSCs”), and culturing the hPSCs in a serum-free media including at least one induction molecule, wherein the at least one induction molecule is capable of generating at least one mesoderm cell. Also decribed herein is a a pharmaceutical composition, including at least one mesoderm cell generated by the method for generating a mesoderm cell, including providing a quantity of human pluripotent stem cells (“hPSCs”), and culturing the hPSCs in a serum-free media including at least one induction molecule, wherein the at least one induction molecule is capable of generating at least one mesoderm cell, and a pharmaceutically acceptable carrier.

Also described herein is a composition of at least one mesenchyme cell generated by the method of for generating a mesoderm cell, including providing a quantity of human pluripotent stem cells (“hPSCs”), and culturing the hPSCs in a serum-free media including at least one induction molecule, wherein the at least one induction molecule is capable of generating at least one mesoderm cell. In other embodiments, the further culturing is in the presence of fibroblast growth factor-9 (“FGF9”) and/or activin A.

Also described herein is an efficient method for generating intermediate mesoderm cells, including providing a quantity of human pluripotent stem cells (“hPSCs”), culturing the hPSCs in a serum-free media comprising CHIR99021 for about 12, 24, 36, or 48 hours, and further culturing in the presence of fibroblast growth factor-2 (“FGF2”) and/or retinoic acid (“RA”) for about 36, 48, 60, or 72 hours, wherein the culturing and further culturing generating intermediate mesoderm cells that express paired box-2 (“PAX2”) and LIM homeobox-1 (“LHX”). In certain embodiments, the method includes further culturing in the presence of fibroblast growth factor-9 (“FGF9”) and/or activin A. In various embodiments, the method generates at least 50%, 60, 70% or more intermediate mesoderm cells.

### DETAILED DESCRIPTION

All references cited herein are incorporated by reference in their entirety as though fully set forth. Unless defined otherwise, technical and scientific terms used herein have the same meaning as commonly understood by one of ordinary skill in the art to which this invention belongs. Allen et al., Remington: The Science and Practice of Pharmacy 22^{nd} ed., Pharmaceutical Press (Sep. 15, 2012); Hornyak et al., Introduction to Nanoscience and Nanotechnology, CRC Press (2008); Singleton and Sainsbury, Dictionary of Microbiology and Molecular Biology 3^{rd} ed., revised ed., J. Wiley & Sons (New York, N.Y. 2006); Smith, March's Advanced Organic Chemistry Reactions, Mechanisms and Structure 7^{th} ed., J. Wiley & Sons (New York, N.Y. 2013); Singleton, Dictionary of DNA and Genome Technology 3rd ed., Wiley-Blackwell (Nov. 28, 2012); and Green and Sambrook, Molecular Cloning: A Laboratory Manual 4th ed., Cold Spring Harbor Laboratory Press (Cold Spring Harbor, N.Y. 2012), provide one skilled in the art with a general guide to many of the terms used in the present application. For references on how to prepare antibodies, see Greenfield, Antibodies A Laboratory Manual 2^{nd} ed., Cold Spring Harbor Press (Cold Spring Harbor N.Y., 2013); Köhler and Milstein, Derivation of specific antibody-producing tissue culture and tumor lines by cell fusion, Eur. J. Immunol. 1976 Jul., 6(7):511-9; Queen and Selick, Humanized immunoglobulins, U.S. Pat. No. 5,585,089 (1996 December); and Riechmann et al., Reshaping human antibodies for therapy, Nature 1988 Mar. 24, 332(6162):323-7.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

As described, few methods have been developed to effect kidney differentiation. While a number of studies have attempted to differentiate mouse ESCs into kidney cells, only a few studies have reported protocols in human ESCs and iPSCs. These previous reports have produced cells which share characteristics expected of human kidney progenitor or epithelial cells. However, the identity of these differentiated cells have yet to be conclusively verified. In addition, the efficiencies of these protocols for generating cells of the renal lineage are low, necessitating the use of cell sorting to enrich populations of cells using markers which are not entirely specific to the kidney. For example, OSR1, a marker used to label cells of the intermediate mesoderm, is also expressed in lateral plate mesoderm, which gives rise during embryonic development to the adult heart, hematopoietic system, and vasculature. Other markers, such as AQP1+ of proximal tubular-like cells, have been suggested, but this marker is expressed not only in the kidney, but also broadly in the gastrointestinal system, lungs, and blood cells. For both markers, sorted cells are heterogeneous and include only a small percentage of cells that exhibited properties and behaviors of cells of the kidney lineage. While existing studies have suggested a role for Wnt, activin, BMP, and retinoic acid signaling in the induction of cells of the kidney lineage, inductive effects of other signaling pathways, such as FGF signaling, on kidney differentiation from hPSCs have not been reported.

Described herein is a method using sequential treatment of CHIR99021 and FGF2 and RA that induces efficient differentiation of hPSCs into PAX2+LHX1+ intermediate mesoderm. The method achieves efficient IM differentiation within 3 days, which is considerably quicker than existing protocols while still maintaining a high level of efficiency. The method is extensible to multiple hESC and hiPSC lines, and importantly, without the need to resort to flow sorting. The resulting PAX2+LHX1+ cells, are capable of autonomous WT1 expression—a later marker of IM differentiation. With the addition of FGF9 and activin, PAX2+LHX1+ cells specifically differentiated into cells expressing SIX2, SALL1, and WT1, markers of cap mesenchyme nephron progenitor cells. Cells generated by the described methods are further capable of forming polarized, ciliated tubular structures express markers of kidney proximal tubular cells and integrate into mouse metanephric cultures. The establishment of this system will facilitate and improve the directed differentiation of hPSCs into cells of the kidney lineage for the purposes of bioengineering kidney tissue and iPS cell disease modeling.

Described herein is a method for generating a mesoderm cell, including providing a quantity of human pluripotent stem cells (“hPSCs”), and culturing the hPSCs in a serum-free media comprising at least one induction molecule, wherein the at least one induction molecule is capable of generating at least one mesoderm cell. In another embodiment, the human pluripotent stem cells are human embryonic stem cell (“hESCs”). In another embodiment, the human pluripotent stem cells are human induced pluripotent stem cells (“hiPSCs”). In another embodiment, the mesoderm cell is an intermediate mesoderm cell. In another embodiment, the intermediate mesoderm cell expresses paired box-2 (“PAX2”), LIM homeobox-1 (“LHX”), and/or Wilms tumor-1 (“WT1”). In another embodiment, the at least one induction molecule is wingless-type MMTV integration site family, member 3A (“WNT3a”) or activin A. In another embodiment, the at least one induction molecule is a Glycogen synthase kinase-3 beta (“GSK3β”) inhibitor. In another embodiment, the GSK3β inhibitor is CHIR99021. In various embodiments, the concentration of CHIR99021 is about 0.5, 1, 2, 3, 4, 5, 6, 7, 8, 9 μM or more. In various embodiments, the concentration of CHIR99021 is about 5 μM. In another embodiment, the hPSCs are cultured in a serum-free media comprising at least one induction molecule for about 12, 24, 36, or 48 hours. In an alternative embodiment, the method includes further culturing of the at least one mesoderm cell in the presence of at least one growth factor. In another embodiment, the at least one growth factor comprises fibroblast growth factor-2 (“FGF2”) and/or retinoic acid (“RA”). In some embodiments, the concentration of FGF2 is about 25, 50, 75, 100, 125, 150, 175, 200 or more ng/mL. In some embodiments, the concentration of FGF2 is about 100 ng/mL. In another embodiment, the concentration of RA is about 0.1, 0.25, 0.5, 1, 2, 3, 4, 5, 6, 7, 8, 9 μM or more. In another embodiment, the concentration of RA is about 1 μM. In another embodiment, further culturing of the at least one mesoderm cell is in the presence of FGF2 and/or RA is for about 36, 48, 60, or 72 hours. In various embodiments, the method does not include the use of a feeder layer.

Further described herein is a composition of at least one mesoderm cell generated by the described method of providing a quantity of hPSCs, and culturing the hPSCs in a serum-free media including at least one induction molecule. Further described herein is a pharmaceutical composition of at least one mesoderm cell generated by the descried method and a pharmaceutically acceptable carrier.

In alternative embodiments, the intermediate mesodermal cells are further cultured to generate metanephric mesenchyme. In various embodiments, the method includes additional culturing of the at least one intermediate mesoderm cell in the presence of at least one further growth factor. In another embodiment, the at least one further growth factor comprises fibroblast growth factor-9 (“FGF9”) and/or activin A. In another embodiment, the concentration of FGF9 is about 25, 50, 75, 100, 125, 150, 175, 200, 300 or more ng/mL. In another embodiment, the concentration of activin A is about 5, 10, 15, 25, 50, 75, 100 or more ng/mL. In another embodiment, further culturing of the at least one mesoderm cell is in the presence of FGF9 and/or activin A is for about 24, 36, 48, 60, 72, 84, or 96 hours. In another embodiment, the at least one intermediate mesoderm cell is a cell that expresses express paired box-2 (“PAX2”) and/or LIM homeobox-1 (“LHX”).

In alternative embodiments, the intermediate mesodermal cells are further cultured to generate a nephrotic cell. In various embodiments, the nephrotic cell expresses Lotus tetragonolobus lectin (“LTL”), kidney-specific protein (“KSP”), and/or the ciliary protein polycystin-2 (“CPP-2”). In alternative embodiments, the nephrotic cell expresses six2 homeobox (“SIX2”), aquaporin-1 or -2 (“AQP1”, “AQP2”), megalin,uromodulin (“UMOD”) In various embodiment, the nephrotic cells possess ciliary structures and/or tubular morphology.

Also described herein is an efficient method for generating intermediate mesoderm cells, including providing a quantity of human pluripotent stem cells (“hPSCs”), and culturing the hPSCs in a serum-free media comprising CHIR99021 for about 12, 24, 36, or 48 hours, and further culturing in the presence of fibroblast growth factor-2 (“FGF2”) and/or retinoic acid (“RA”) for about 36, 48, 60, or 72 hours, wherein the culturing and further culturing generating intermediate mesoderm cells that express paired box-2 (“PAX2”) and LIM homeobox-1 (“LHX”). In various embodiments, the concentration of CHIR99021 is about 5 μM, the concentration of FGF2 is about 100 ng/mL, and the concentration of RA is about 1 μM. In another embodiment, the method generates at least 50%, 60, 70% or more intermediate mesoderm cells.

In addition, described herein is a method for generating a mesoendoderm cell, including providing a quantity of human pluripotent stem cells (“hPSCs”), and culturing the hPSCs in a serum-free media comprising at least one induction molecule, wherein the at least one induction molecule is capable of generating at least one mesoendoderm cell. In another embodiment, the human pluripotent stem cells are human embryonic stem cell (“hESCs”). In another embodiment, the human pluripotent stem cells are human induced pluripotent stem cells (“hiPSCs”). In another embodiment, the mesoendoderm cell is capable of differentiating into an mesoderm or endoderm cell. In another embodiment, the mesoendoderm cell expresses BRACHYURY.

In another embodiment, the mesoendoderm cell is capable of forming a definitive endoderm cell. In another embodiment, the definitive endoderm cell expresses sry homology box-17 (“SOX17”). In various embodiment, the mesoderm cell does not express sry homology box-1 (“SOX1”) and/or or paired box-2 (“PAX6”)

In another embodiment, the mesoenderm cell is capable of forming a mesoderm cell. In another embodiment, the mesoderm cell expresses forkheadbox-1 (“FOXF1”), kinase domain receptor (“KDR”), t-box-6 (“TBX6”), and/or paired box-2 (“PAX2”). In various embodiment, the mesoderm cell does not express sry homology box-1 (“SOX1”) and/or paired box-2 (“PAX6”)

In another embodiment, the mesoendoderm cell is capable of forming an intermediate mesoderm cell. In another embodiment, the intermediate mesoderm cell expresses paired box-2 (“PAX2”), LIM homeobox-1 (“LHX”), and/or Wilms tumor-1 (“WT1”). In another embodiment, the at least one induction molecule is wingless-type MMTV integration site family, member 3A (“WNT3a”) or activin A. In another embodiment, the at least one induction molecule is a Glycogen synthase kinase-3 beta (“GSK3β”) inhibitor. In another embodiment, the GSK3β inhibitor is CHIR99021. In various embodiments, the concentration of CHIR99021 is about 0.5, 1, 2, 3, 4, 5, 6, 7, 8, 9 μM or more. In various embodiments, the concentration of CHIR99021 is about 5 μM. In another embodiment, the hPSCs are cultured in a serum-free media comprising at least one induction molecule for about 12, 24, 36, or 48 hours. In an alternative embodiment, the method includes further culturing of the at least one mesoderm cell in the presence of at least one growth factor. In another embodiment, the at least one growth factor comprises fibroblast growth factor-2 (“FGF2”) and/or retinoic acid (“RA”). In some embodiments, the concentration of FGF2 is about 25, 50, 75, 100, 125, 150, 175, 200 or more ng/mL. In some embodiments, the concentration of FGF2 is about 100 ng/mL. In another embodiment, the concentration of RA is about 0.1, 0.25, 0.5, 1, 2, 3, 4, 5, 6, 7, 8, 9 μM or more. In another embodiment, the concentration of RA is about 1 μM. In another embodiment, further culturing of the at least one mesoderm cell is in the presence of FGF2 and/or RA is for about 36, 48, 60, or 72 hours. In various embodiments, the method does not include the use of a feeder layer.

In another embodiment, the intermediate mesoderm cell is capable of forming metanephric mesenchyme. In another embodiment, the metanephric mesenchyme expresses (“PAX2”), LIM homeobox-1 (“LHX”), Wilms tumor-1 (“WT1”), and/or six2 homeobox (“SIX2”). In another embodiment, the at least one induction molecule is WNT3a or activin A. In another embodiment, the at least one induction molecule is a Glycogen synthase kinase-3 beta (“GSK3β”) inhibitor. In another embodiment, the GSK3β inhibitor is CHIR99021. In another embodiment, the hPSCs are cultured in a serum-free media comprising at least one induction molecule for about 12, 24, 36, or 48 hours. In an alternative embodiment, the method includes further culturing of the at least one mesoderm cell in the presence of at least one growth factor. In another embodiment, the at least one growth factor comprises fibroblast growth factor-2 (“FGF2”) and/or retinoic acid (“RA”). In another embodiment, further culturing of the at least one mesoderm cell is in the presence of FGF2 and/or RA is for about 36, 48, 60, or 72 hours. In an alternative embodiment, the method includes additional culturing of the at least one intermediate mesoderm cell in the presence of at least one further growth factor. In another embodiment, the at least one further growth factor comprises fibroblast growth factor-9 (“FGF9”) and/or activin A. In another embodiment, the concentration of FGF9 is about 25, 50, 75, 100, 125, 150, 175, 200, 300 or more ng/mL. In another embodiment, the concentration of activin A is about 5, 10, 15, 25, 50, 75, 100 or more ng/mL. In another embodiment, further culturing of the at least one mesoderm cell is in the presence of FGF9 and/or activin A is for about 24, 36, 48, 60, 72, 84, or 96 hours. In another embodiment, the at least one intermediate mesoderm cell is a cell that expresses express paired box-2 (“PAX2”) and/or LIM homeobox-1 (“LHX”). In various embodiments, the method does not include the use of a feeder layer.

### Example 1

### Human ES and iPS Cell Culture

For generation of human iPS cells, BJ (ATCC) and HDFalpha (Invitrogen) fibroblasts can be reprogrammed by two rounds of overnight transduction with pMIG retroviruses for OCT4, SOX2, KLF4, and c-MYC (Addgene) produced in 293FT cells (Invitrogen). Human embryonic stem cell lines, H1, H9, and CHB8-H2B-GFP hESCs (passages 30-50), as well as BJ and HDF iPSCs (passages 12-40) can be cultured on irradiated mouse embryonic fibroblasts (GlobalStem) in DMEM/F12 (Invitrogen) supplemented with 20% KnockOut serum replacement (Invitrogen), 1 mM nonessential amino acids (Invitrogen), 2 mM Glutamax (Invitrogen), 0.55 mM 2-mercaptoethanol (Invitrogen), penicillin/streptomycin (Invitrogen), and 10 ng/mL recombinant human bFGF/FGF2 (Invitrogen).

Cells can be passaged using Collagenase Type IV (STEMCELL Technologies) at a 1:3 split ratio every 5-7 d. For feeder-free culture, hESCs previously grown on MEFs are initially passaged using Collagenase Type IV onto plates coated with Geltrex hESC-qualified reduced growth factor basement membrane matrix (Invitrogen) according to manufacturer's instructions and cultured in either mTeSR1 medium (STEMCELL Technologies) supplemented with penicillin/streptomycin or ReproFF2 medium (ReproCELL) supplemented with FGF2.

### Example 2

### Differentiation

For differentiation experiments, hESCs or hiPSCs can be grown on Geltrex, washed once with PBS and dissociated into single cells with Accutase (STEMCELL Technologies). Cells are plated at a density of 4×10⁴ cells/cm² onto Geltrex-coated plates in mTeSR1 medium supplemented with the ROCK inhibitor Y27632 10 μM (Stemgent). Cells are fed daily with mTeSR1 without Y27632 for 2-3 days until they reached 50% confluency.

Mesendoderm differentiation—the media is changed to Advanced RPMI (A-RPMI, Invitrogen) supplemented with 1×L-glutamax and 1× penicillin/streptomycin and either CHIR99021 (CHIR, Stemgent), human Wnt3a (R&D systems), and/or human activin A (R&D systems) at the doses described.
Definitive endoderm differentiation—cells are treated with A-RPMI+1×L-glu+1×P/S+5 μM CHIR for 24 hours, then A-RPMI+1×Lglu+1×P/S+100 ng/mL of activin A for 2-3 days.
Hepatic differentiation—cells at the definitive endoderm stage are treated with A-RPMI+1×L-glu+1×P/S+1×B27 supplement (Invitrogen)+20 ng/mL BMP-4 (R&D systems)+10 ng/mL FGF2 (Invitrogen) for 5 days, then A-RPMI+1×L-glu+1×P/S+1×B27+10 ng/mL HGF for 5 days, then HCM Hepatocyte Culture Medium (Lonza) supplemented with 20 ng/mL Oncostatin M and 10 ng/mL HGF (Peprotech) for 5 days.
Pancreatic differentiation—cells at the definitive endoderm stage are treated with DMEM/F12+2% FBS (Hyclone)+50 ng/mL FGF7 (R&D systems) for 2 days, then high-glucose DMEM (Mediatech)+1% B27+2 μM retinoic acid (Sigma)+0.25 μM KAAD cyclopamine (EMD Millipore)+100 ng/mL recombinant human Noggin (R&D systems) for 4 days, then high-glucose DMEM+1% B27+100 ng/mL Noggin+300 nM indolactam V (Stemgent)+1 μM ALK5 inhibitor II (Axxora) for 4 days.
Anterior foregut endoderm differentiation—cells at the definitive endoderm stage are treated with DMEM/F12+1×L-glu+1×B27+200 ng/mL Noggin+10 μM SB431542 (Stemgent) for 3 days. For hindgut endoderm differentiation, cells at the definitive endoderm stage were treated with A-RPMI+1×L-glu+1×P/S+2% FBS+500 ng/mL FGF4 (R&D systems)+5 μM CHIR for 4 days.
Hindgut endoderm differentiation—cells at the definitive endodermstage were treated with A-RPMI+13 L-glu+13 P/S+2% FBS+500 ng/ml FGF4 (R&D Systems)+5mMCHIR for 4 days.
Intermediate mesoderm differentiation—cells are treated with A-RPMI+1×L-glu+1×P/S+5 μM CHIR for 24, 36, or 48 hours, then A-RPMI+1×L-glu+1×P/S+100 ng/mL FGF2+1 μM retinoic acid for 2-3 days.
Cap mesenchyme differentiation—cells were treated with A-RPMI+13 L-glu+13 P/S+5 mM CHIR for 36 hours, then A-RPMI+13 L-glu+13 P/S+100 ng/ml FGF2+1 mM retinoic acid for 36-42 hours, then A-RPMI+13L-glu+13P/S+100 ng/ml FGF-9 (R&D Systems)+10 ng/ml activin A for 3 days.

### Example 3

### Characterization of Differentiated Cells Via Immunofluorescence

For immunofluorescence studies, cultures are washed once with PBS (Invitrogen) and fixed in 4% paraformaldehyde for 15 min at room temperature (RT). Fixed cells are then washed three times in PBS and incubated in blocking buffer (0.3% Triton X-100 (Fisher Scientific) and 5% normal donkey serum (EMD Millipore) in PBS) for 1 hour at RT.

The cells are then incubated with primary antibody overnight at 4° C. or for 2 hours at RT in antibody dilution buffer (0.3% Triton X-100 and 1% BSA (Roche) in PBS). Cells are then washed three times in PBS and incubated with Alexa Fluor 488-, 555-, or 647-conjugated secondary antibodies (1:500) (Molecular Probes) in antibody dilution buffer for 1 hour at RT. For immunostaining with Biotinylated Lotus Tetragonolobus Lectin (LTL, Vector Labs), a Streptavidin/Biotin Blocking Kit (Vector Labs) and Alexa Fluor 488- or 647-conjugated Streptavidin (Molecular Probes) were used according to manufacturer's instructions.

Nuclei can be counterstained with DAPI (Sigma). A list of primary antibodies can be found in Table 1. Immunofluorescence is visualized using an inverted fluorescence microscope (Nikon Eclipse Ti, Tokyo, Japan). Quantification is performed by counting a minimum of five random fields at 10× magnification.

TABLE 1
Antibodies Used in Differentiation Studies

| Antibody | Source | Dilution |
|---|---|---|
| Acetylated Tubulin | Sigma | 1:500 |
| AFP | Dako | 1:200 |
| Albumin | Dako | 1:200 |
| BRACHURY | Santa Cruz | 1:100 |
| CDX2 | Biogenex | 1:100 |
| CK19 | Dako | 1:50 |
| E-cadherin | Abeam | 1:1000 |
| FOXA2 | EMD Millipore | 1:200 |
| FOXF1 | R&D | 1:500 |
| HNF4A | Santa Cruz | 1:100 |
| Human Nuclear Antigen | Millipore | 1:250 |
| Insulin | Dako | 1:1000 |
| Kidney-specific protein | Gift from Dr. Hiroshi Itoh | 1:100 |
| | and Dr. Toshiaki Monkawa | |
| Laminin | Sigma | 1:500 |
| LHX1 | Developmental Studies | 1:50 |
| | Hybridoma Bank | |
| Lotus Tetragonolobus | Vector Labs | 1:200 |
| Lectin (LTL), | | |
| biotinylated | | |
| MIXL1 | Gift from Dr. Andrew Elefanty | 1:100 |
| N-cadherin | Abeam | 1:100 |
| OCT4 | Santa Cruz | 1:100 |
| PAX2 | Covance | 1:100 |
| PAX6 | Stemgent | 1:100 |
| PDX1 | R&D | 1:100 |
| Pro-insulin C-peptide | EMD Millipore | 1:500 |
| Polycystin-2 | Santa Cruz | 1:100 |
| Somatostatin | Dako | 1:1000 |
| SOX2 | Santa Cruz | 1:100 |
| SOX17 | R&D | 1:100 |
| TBX6 | R&D | 1:100 |

### Example 4

### Characterization of Differentiated Cells Via Quantitative RT-PCR

Total RNA is harvested and isolated from cells using the RNeasy Mini Kit (Qiagen). 1 μg RNA is used for reverse transcription with the M-MLV Reverse Transcriptase system (Promega), or 500 ng RNA is used for High Capacity cDNA Reverse Transcription Kits (Applied Biosystems). RT-PCR reactions can be run in duplicate using cDNA (diluted 1:10), 300 or 400 nM forward and reverse primers, and iQ SYBR Green Supermix (Biorad) or iTAQ SYBR Green Supermix (Biorad). Quantitative RT-PCR is performed using the iQ5 Multicolor Real-Time PCR Detection System (Biorad). Samples can be run with two technical replicates to ensure precision and accuracy. β-actin can be used as a housekeeping gene. Primer sequences are listed in Table 2.

TABLE 2
Primers Used in qRT-PCT Differentiation Studies

| Gene | Forward | Reverse |
|---|---|---|
| AQP1 | ATTAACCCTGCTCGGTCCTT | ACCCTGGAGTTGATGTCGTC |
| | [SEQ ID NO: 1] | [SEQ ID NO: 2] |
| AQP2 | ACGCCTTCACGTGTGTGTAT | TTGTTTTCTGCGCCGAAGTG |
| | [SEQ ID NO: 3] | [SEQ ID NO: 4] |
| B-ACTIN | CCAACCGCGAGAGAGTGA | TCCATCACGATGCCAGTG |
| | [SEQ ID NO: 5] | [SEQ ID NO: 6] |
| BMP-4 | AAGCGTAGCCCTAAGCATCA | TGGTTGAGTTGAGGTGGTCA |
| | [SEQ ID NO: 7] | [SEQ ID NO: 8] |
| BRACHYURY | GTGCTGTCCCAGGTGGCTTACAGATG | CCTTAACAGCTCAACTCTAACTACTTG |
| | [SEQ ID NO: 9] | [SEQ ID NO: 10] |
| CXCR4 | CACCGCATCTGGAGAACCA | GCCCATTTCCTCGGTGTAGTT |
| | [SEQ ID NO: 11] | [SEQ ID NO: 12] |
| EOMES | ATCATTACGAAACAGGGCAGGC | CGGGGTTGGTATTTGTGTAAGG |
| | [SEQ ID NO: 13] | [SEQ ID NO: 14] |
| EYA1 | TGCATATGGGCAAACACAGT | CCAGGTTGAGGGGTACTGAA |
| | [SEQ ID NO: 15] | [SEQ ID NO: 16] |
| EYA2 | CCGGTCTAAGAGGAGCAGTG | CTGGTCACAATCCTCCAGGT |
| | [SEQ ID NO: 17] | [SEQ ID NO: 18] |
| FOXA2 | CCATTGCTGTTGTTGCAGGGAAGT | CACCGTGTCAGGATTGGGAATGCT |
| | [SEQ ID NO: 19] | [SEQ ID NO: 20] |
| FOXF1 | CAGCCTCACATCACGCAAGG | AGCCGAGCTGCAAGGCATC |
| | [SEQ ID NO: 21] | [SEQ ID NO: 22] |
| FOXI3 | CCACCCCTTGTCTCAACACT | TTGCTCAGTTGCAAGGTGTC |
| | [SEQ ID NO: 23] | [SEQ ID NO: 24] |
| GSC | GAGGAGAAAGTGGAGGTCTGGTT | CTCTGATGAGGACCGCTTCTG |
| | [SEQ ID NO: 25] | [SEQ ID NO: 26] |
| KDR | TTTTTGCCCTTGTTCTGTCC | TCATTGTTCCCAGCATTTCA |
| | [SEQ ID NO: 27] | [SEQ ID NO: 28] |
| LHX1 | ATCCTGGACCGCTTTCTCTT | GTACCGAAACACCGGAAGAA |
| | [SEQ ID NO: 29] | [SEQ ID NO: 30] |
| MEGALIN | TGTGATGCAGCCATCGAACT | TGCATTTGGGGAGGTCAGTC |
| | [SEQ ID NO: 31] | [SEQ ID NO: 32] |
| MEOX1 | AAAGTGTCCCCTGCATTCTG | CACTCCAGGGTTCCACATCT |
| | [SEQ ID NO: 33] | [SEQ ID NO: 34] |
| MIXL1 | ACGTCTTTCAGCGCCGAACAG | TTGGTTCGGGCAGGCAGTTCA |
| | [SEQ ID NO: 35] | [SEQ ID NO: 36] |
| NANOG | TGCTTATTCAGGACAGCCCT | TCTGGTCTTCTGTTTCTTGACT |
| | [SEQ ID NO: 37] | [SEQ ID NO: 38] |
| OCT4 | CAGTGCCCGAAACCCACAC | GGAGACCCAGCAGCCTCAAA |
| | [SEQ ID NO: 39] | [SEQ ID NO: 40] |
| OSR1 | CCTTCCTTCAGGCAGTGAAC | CGGCACTTTGGAGAAAGAAG |
| | [SEQ ID NO: 41] | [SEQ ID NO: 42] |
| OTX2 | GCAGAGGTCCTATCCCATGA | CTGGGTGGAAAGAGAAGCTG |
| | [SEQ ID NO: 43] | [SEQ ID NO: 44] |
| PAX2 | CAAAGTTCAGCAGCCTTTCC | CCACACCACTCTGGGAATCT |
| | [SEQ ID NO: 45] | [SEQ ID NO: 46] |
| PAX6 | ACCCATTATCCAGATGTGTTTGCCCGAG | ATGGTGAAGCTGGGCATAGGCGGCAG |
| | [SEQ ID NO: 47] | [SEQ ID NO: 48] |
| PAX8 | GCAACCATTCAACCTCCCTA | CTGCTGCTGCTCTGTGAGTC |
| | [SEQ ID NO: 49] | [SEQ ID NO: 50] |
| SIX2 | CTGGAGAGCCACCAGTTCTC | GCTGCGACTCTTTTCCTTGA |
| | [SEQ ID NO: 51] | [SEQ ID NO: 52] |
| SIX4 | CCCAAGATGGAGGGTCTGTA | TGTGCTTCCATCTGAAGTGC |
| | [SEQ ID NO: 53] | [SEQ ID NO: 54] |
| SOX1 | CAATGCGGGGAGGAGAAGTC | CTCTGGACCAAACTGTGGCG |
| | [SEQ ID NO: 55] | [SEQ ID NO: 56] |
| SOX3 | AGACCAGGACCGTGTGAAAC | GTCGATGAATGGTCGCTTCT |
| | [SEQ ID NO: 57] | [SEQ ID NO: 58] |
| SNAI1 | CCCACATCCTTCTCACTGC | GTCAGCCTTTGTCCTGTAGC |
| | [SEQ ID NO: 59] | [SEQ ID NO: 60] |
| SOX17 | CGCACGGAATTTGAACAGTA | GGATCAGGGACCTGTCACAC |
| | [SEQ ID NO: 61] | [SEQ ID NO: 62] |
| TBX6 | AAGTACCAACCCCGCATACA | TAGGCTGTCACGGAGATGAA |
| | [SEQ ID NO: 63] | [SEQ ID NO: 64] |
| UMOD | AAACCCATGCCACTTACAGC | CGGTCTTCAGGCTGACTTTC |
| | [SEQ ID NO: 65] | [SEQ ID NO: 66] |
| WT1 | GGGTACGAGAGCGATAACCA | TCTCACCAGTGTGCTTCCTG |
| | [SEQ ID NO: 67] | [SEQ ID NO: 68] |

### Example 5

### Flow Cytometry

Cells are dissociated using Accutase for 15 minutes, and cell clumps are removed with a 40 μm cell strainer (BD Biosciences). Cells are fixed with 2% PFA for 15 minutes on ice and then permeabilized with 0.1% Triton for 15 minutes on ice. Cells are blocked with PBS+5% donkey serum for 15 minutes on ice and incubated with primary antibodies (PAX2 1:1000, LHX1 1:100) for 30 minutes. After washing 3 times with 1% BSA in PBS, cells were incubated with secondary antibodies (Alexa Fluor 488-conjugated donkey antirabbit 1:5000, Cy5-conjugated donkey anti-mouse 1:2500 (Jackson ImmunoResearch)) for 20 minutes on ice. Cells are then washed three times with 1% BSA in PBS. Flow cytometry is performed using MACSQuant (Miltenyi Biotec), and data analysis is performed using FlowJo software. Optimal dilution ratios of antibodies is determined using a negative control human proximal tubular cell line (HKC-8) which does not express PAX2 or LHX1.

### Example 6

### Whole-Mount Immunohistochemistry of Cultured Kidneys

Immunochemistry protocol is based on previous methods for kidney explant cultures. Filters with cultured explants were rinsed once with PBS then fixed with 4% PFA in PBS for 30 minutes in a 24-well plate. Care was taken to submerge the explants to ensure even fixation. Following fixation, explants were washed three times in PBT (PBS with 0.1% Triton X-100) for 5 minutes each at room temperature with gentle rocking Explants were then incubated in blocking solution (PBT with 5% donkey serum) at room temperature with rocking Blocking solution was then removed and replaced with a primary antibody solution of diluted antibodies in PBT with 1% donkey serum and samples incubated overnight at 4° C. Antibody dilutions used: mouse anti-Human Nuclear Antigen (HNA), 1:250 (Millipore); rabbit anti-laminin 1:500 (Sigma). Explants were then washed with PBT three times for one hour each with rocking at room temperature. Secondary antibody solution (PBT+1% donkey serum) with 1:250 dilutions of Alexa Fluor 488-conjugated donkey anti-mouse and Alexa Fluor 568-conjugated donkey anti-rabbit antibodies (Invitrogen) were added and incubated for 1-2 hours at room temperature. Samples were then washed with PBT three times for 30 minutes each at room temperature, followed by a 10 minute incubation with DAPI, and three additional 5 minute washes with PBS. Explant samples where then mounted with Vectashield (Vector Labs) and examined using a Nikon C1 confocal microscope.

### Example 7

### CHIR99021 Efficiently Induces Human Pluripotent Stem Cells to Differentiate into Mesendoderm

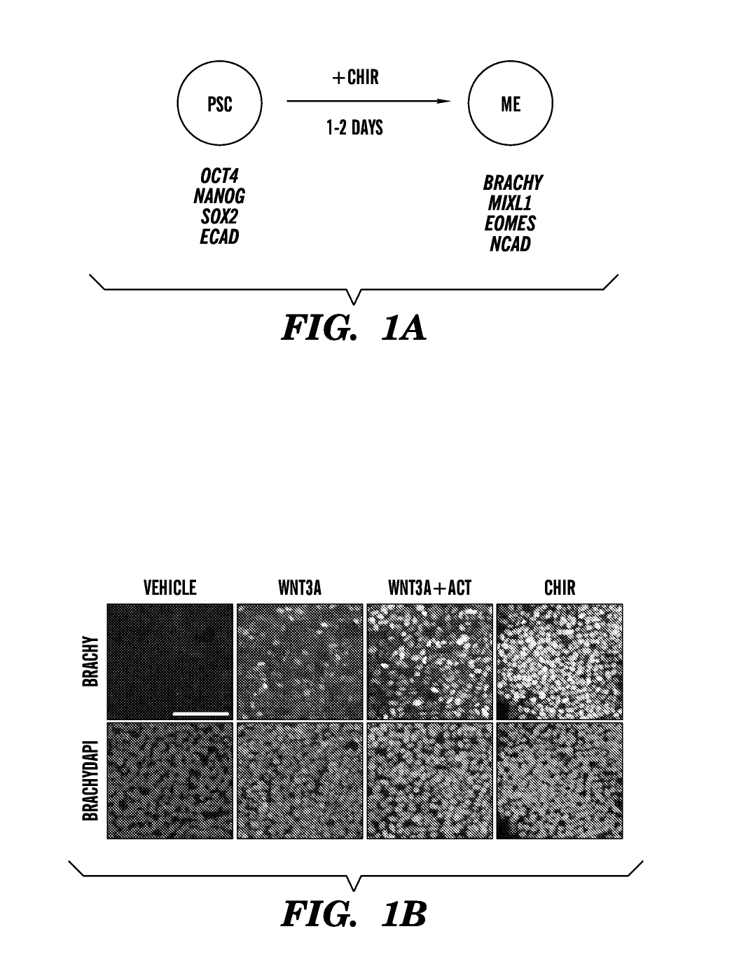

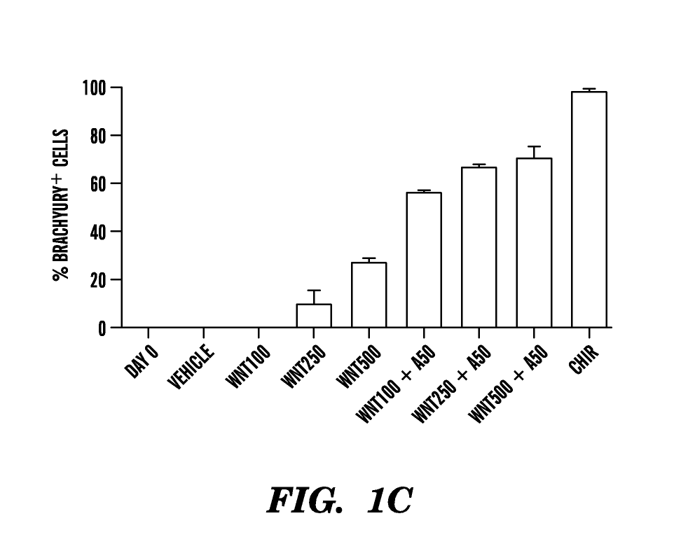

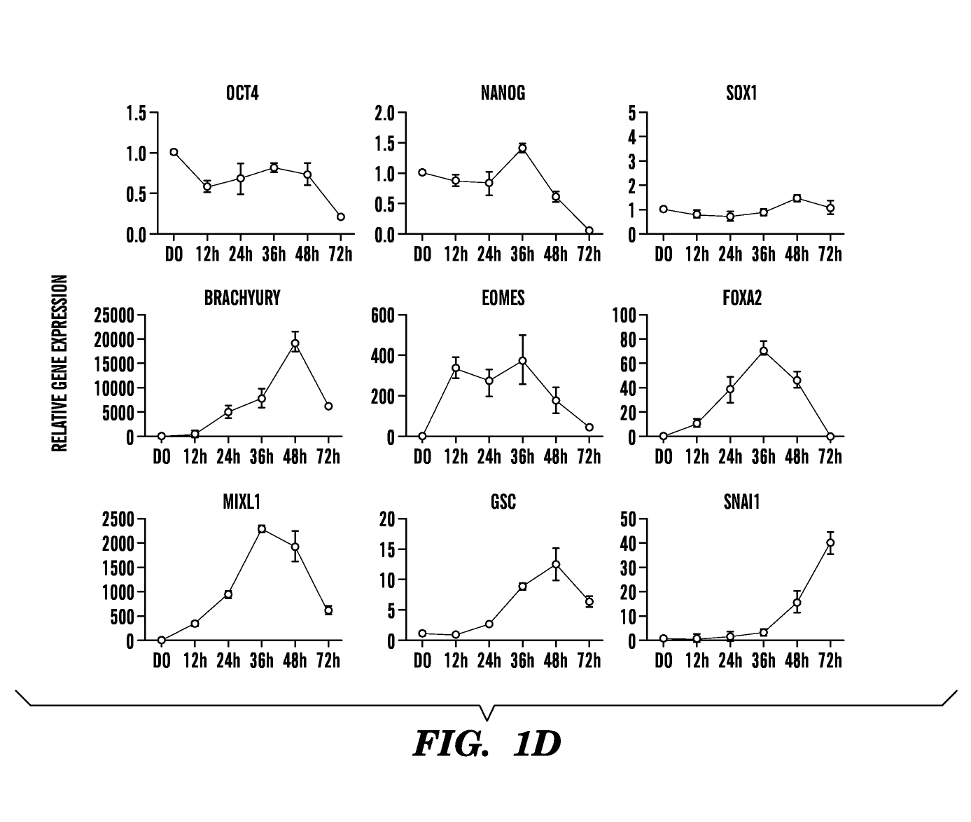

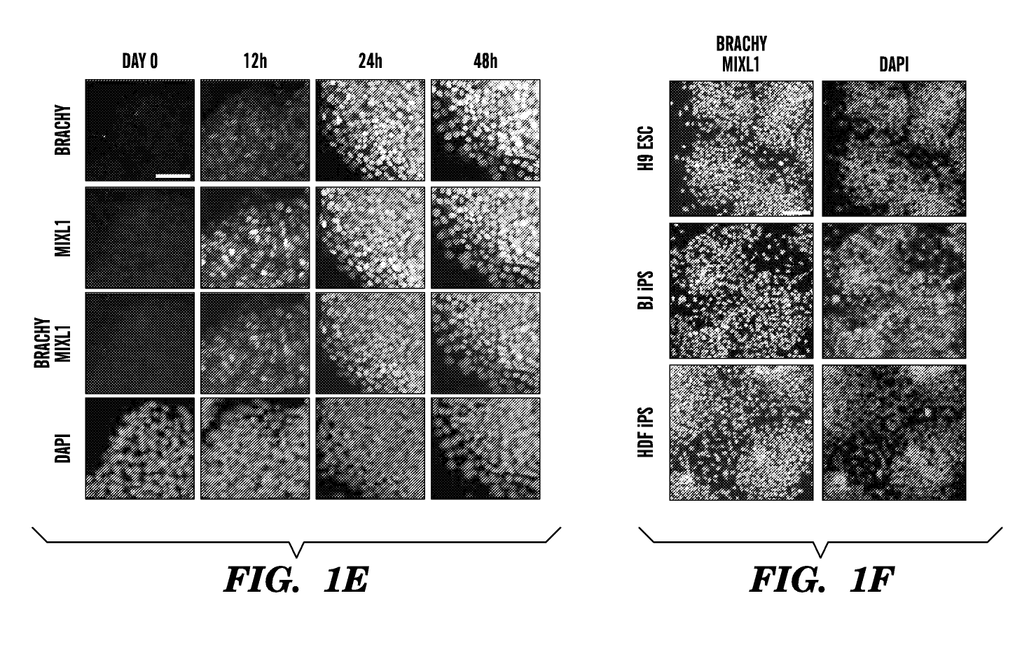

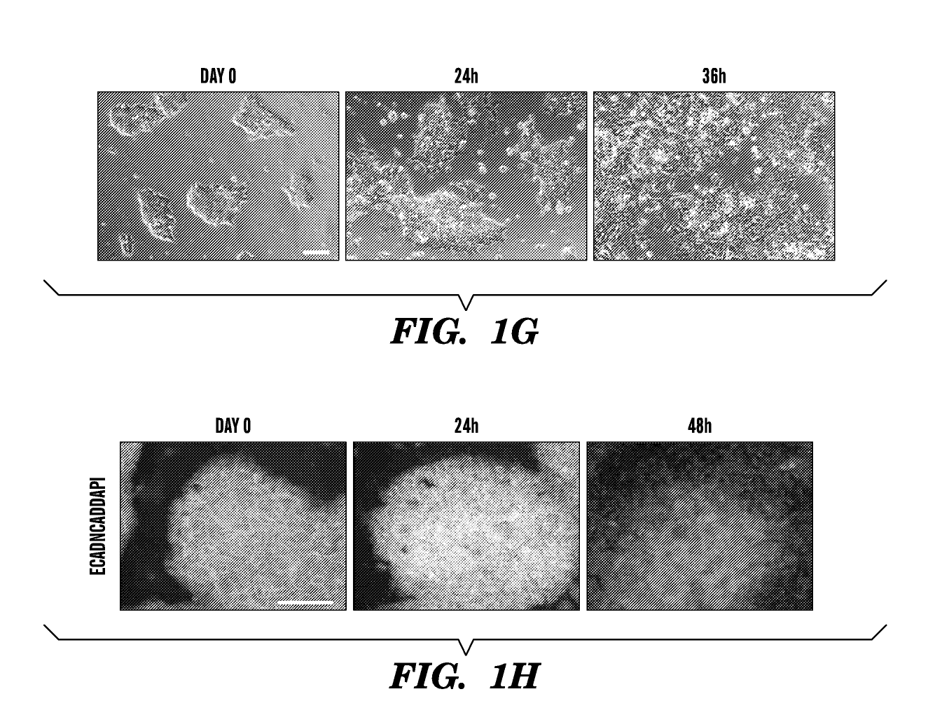

To develop a platform to target generation of IM, conditions most efficiently differentiate hPSCs into mesendoderm must first be identified (FIG. 1A). During gastrulation, cells of the epiblast ingress through the primitive streak to form the endodermal and mesodermal germ layers of the developing embryo, and this process is dependent upon the secreted molecules of the Wnt and nodal/activin signaling pathways. To determine the minimal signals required to induce the mesendoderm state in hPSCs, the Inventors investigated differentiated cells under serum-free, feeder-free conditions in the absence of other chemical additives except for the precise signals tested.

An initial screen tested efficacy of WNT3A and activin A to induce expression of the primitive streak marker BRACHYURY (BRACHY). WNT3A alone, at a concentration of 500 ng/mL, induced BRACHY expression in 26.7±1.9% of cells after 24 hours of treatment (FIG. 1B,C). The addition of activin A to WNT3A increased the percentage of BRACHY+ cells to 70.3±5.5% of the total population. However, when cells were treated with 5 μM CHIR99021 (CHIR), a potent GSK3β inhibitor/Wnt pathway agonist, 98.7±1.3% of cells expressed BRACHY at 24 hours (FIG. 1B,C).

Gene expression profiling of CHIR-induced cells revealed that expression of primitive streak genes (BRACHY, MIXL1, EOMES, FOXA2, and GSC) was rapidly upregulated within 24 hours of treatment, peaked between 36-48 hours, and decreased by 72 hours, a pattern consistent with the transient expression of these genes during gastrulation. In parallel, the induction of the mesendoderm state was accompanied by the loss of pluripotency as reflected by a reduction of OCT4 and NANOG mRNA (FIG. 1D). Protein levels paralleled this gene expression pattern, as immunostaining for BRACHY and MIXL1 over the first 48 hours of differentiation revealed co-expression of these markers in CHIR-induced cells as early as 12 hours with co-expression peaking at 48 hours (FIG. 1E,F). As cells migrate through the primitive streak during development, they express SNAIL, downregulate E-cadherin expression, upregulate N-cadherin expression, and undergo an epithelial-mesenchymal transition (“EMT”). Consistent with these observations, CHIR induction resulted in the upregulation of SNAI1 as early as 24 hours after treatment (FIG. 1D). This was associated with EMT with waves of cells migrating away from the periphery of differentiating colonies between 36-48 hours of differentiation (FIG. 1G), and a switch in cadherin expression from E-cadherin to N-cadherin (FIG. 1H). Together these findings demonstrated the potency of CHIR to induce mesendoderm differentiation in hPSCs via a program that mimics normal development in vivo.

### Example 8

### Timed Addition of Exogenous Factors Modulates Cell Fate of CHIR-Induced hPSCs

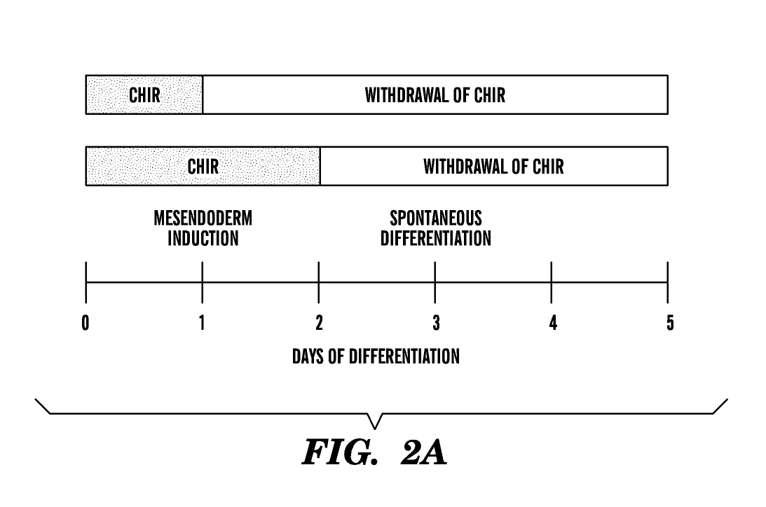

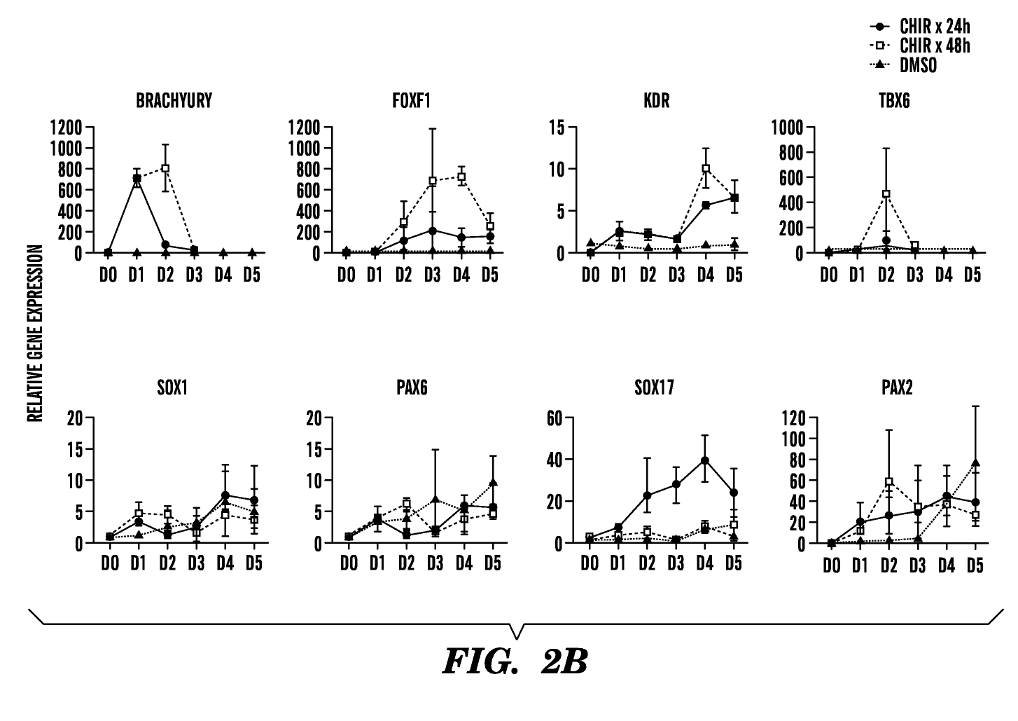

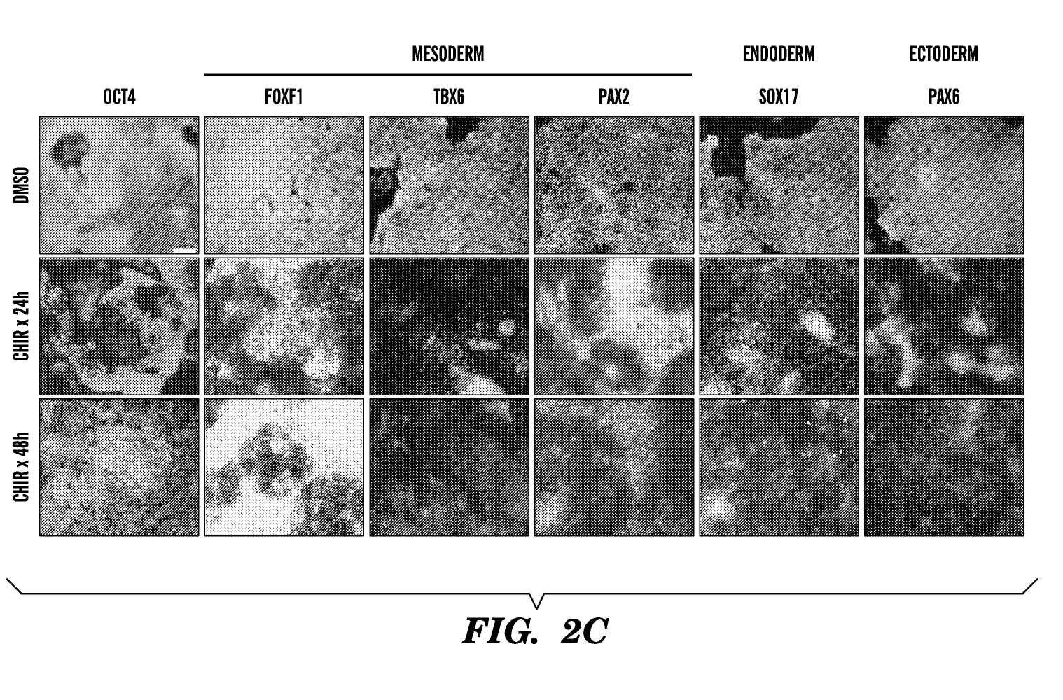

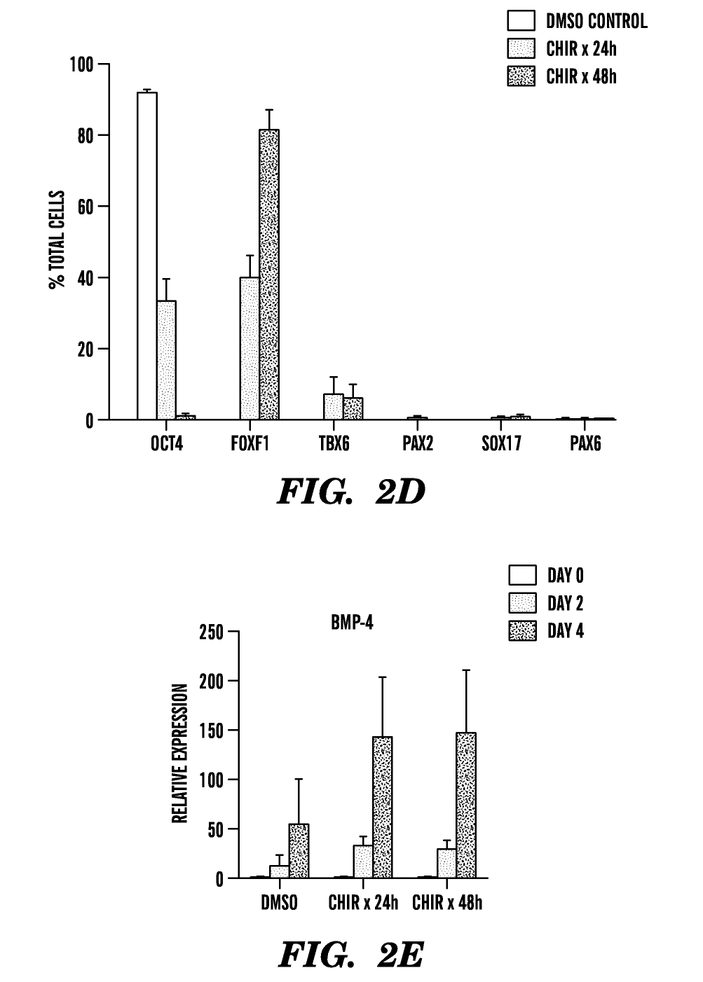

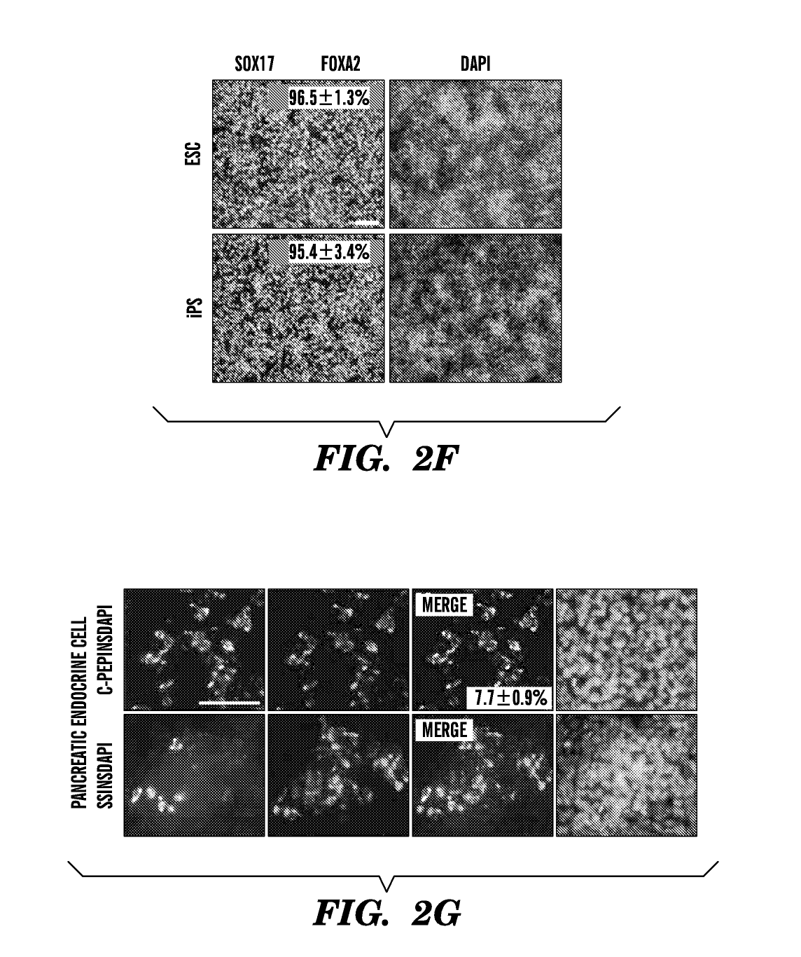

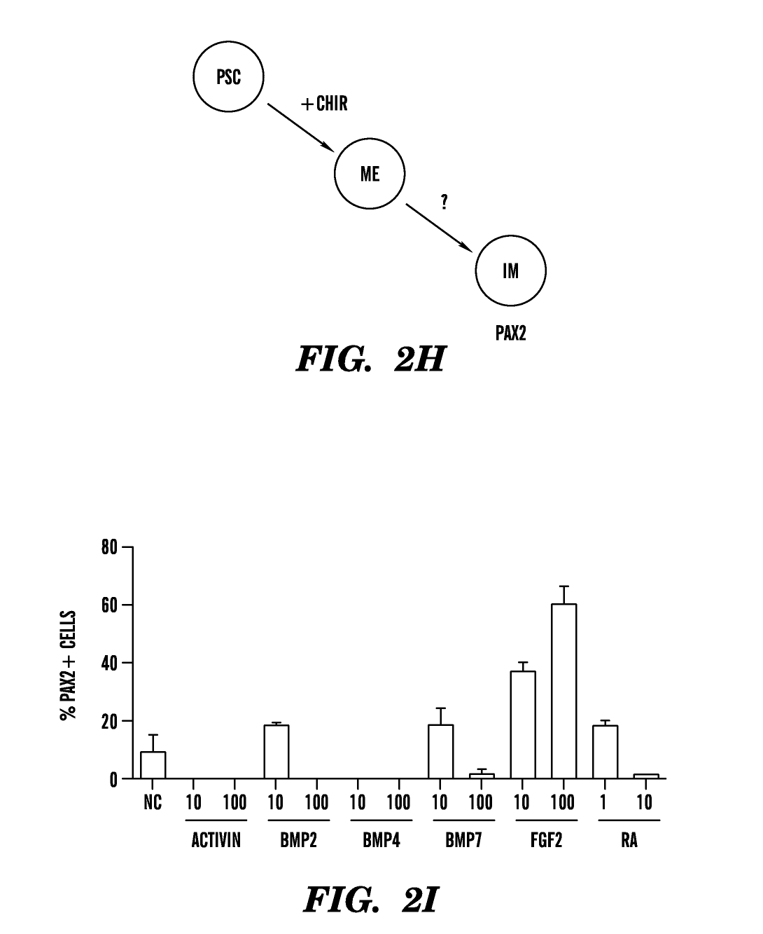

To determine whether treatment with CHIR alone is sufficient to induce differentiation towards IM, the Inventors investigated the intrinsic multipotency of CHIR-induced mesendodermal cells by withdrawing CHIR from the culture media after either 24 or 48 hours of induction and allowing the cells to differentiate for another 3-4 days (FIG. 2A). CHIR-induced mesendodermal cells spontaneously differentiated into heterogeneous populations of cells expressing transcriptional markers of definitive endoderm (SOX17) and mesodermal subtypes (FOXF1, KDR, TBX6, PAX2) without significant upregulation of neuroectodermal markers (SOX1, PAX6) (FIG. 2B). Immunocytochemistry of these cell cultures revealed a dependence of cell fate upon the duration of CHIR treatment. hPSCs pulsed with CHIR for 24 hours generated approximately 40% FOXF1+ lateral plate mesoderm (FIG. 2C,D). A significant proportion of cells (30-40%), however, expressed the pluripotency marker OCT4 (FIG. 2C,D), suggesting that the withdrawal of CHIR after 24 hours may have resulted in the reversion of incompletely differentiated cells back to the pluripotent state. Prolonging the duration of CHIR treatment to 48 hours eliminated the appearance of OCT4+ cells, with greater than 80% of cells staining positive for FOXF1. The protein expression of the paraxial mesoderm marker TBX6 and the IM marker PAX2 was seen in fewer than 10% and 1% of cells, respectively, signifying that CHIR treatment was insufficient to efficiently induce these mesodermal subtypes (FIG. 2C). Similarly, fewer than 1% of cells expressed either SOX17 or PAX6, suggesting that endodermal or ectodermal differentiation was not significantly induced by CHIR treatment.

### Example 9

### BMP-4 Signaling in Lateral Plate Mesoderm Formation

As proper differentiation of lateral plate mesoderm during embryonic development is dependent on higher BMP-4 signaling gradients, the Inventors investigated the expression of BMP-4 transcripts in CHIR-induced cells by quantitative RT-PCR. hPSCs treated with CHIR for 24 or 48 hours significantly upregulated BMP-4 on day 4 compared to DMSO-treated controls, demonstrating that induction with CHIR stimulated endogenous expression of BMP-4 (FIG. 2E).

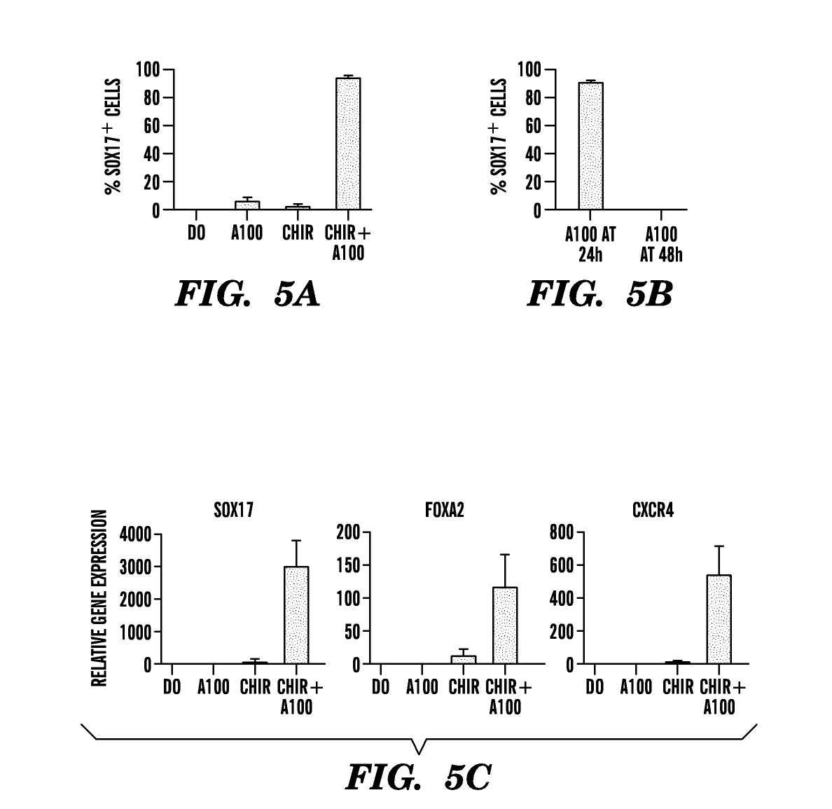

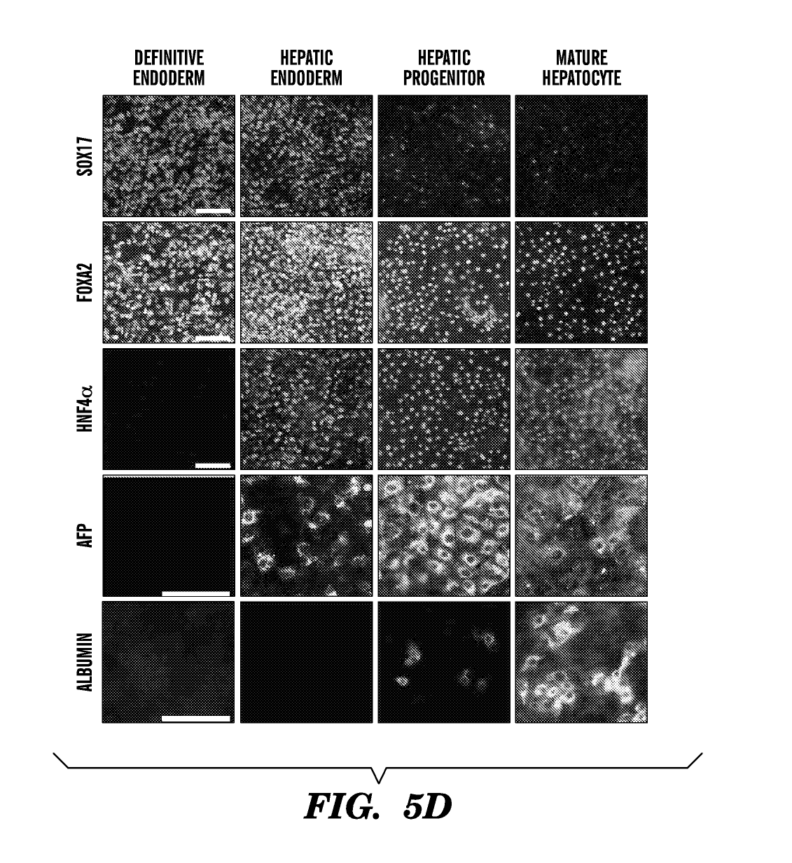

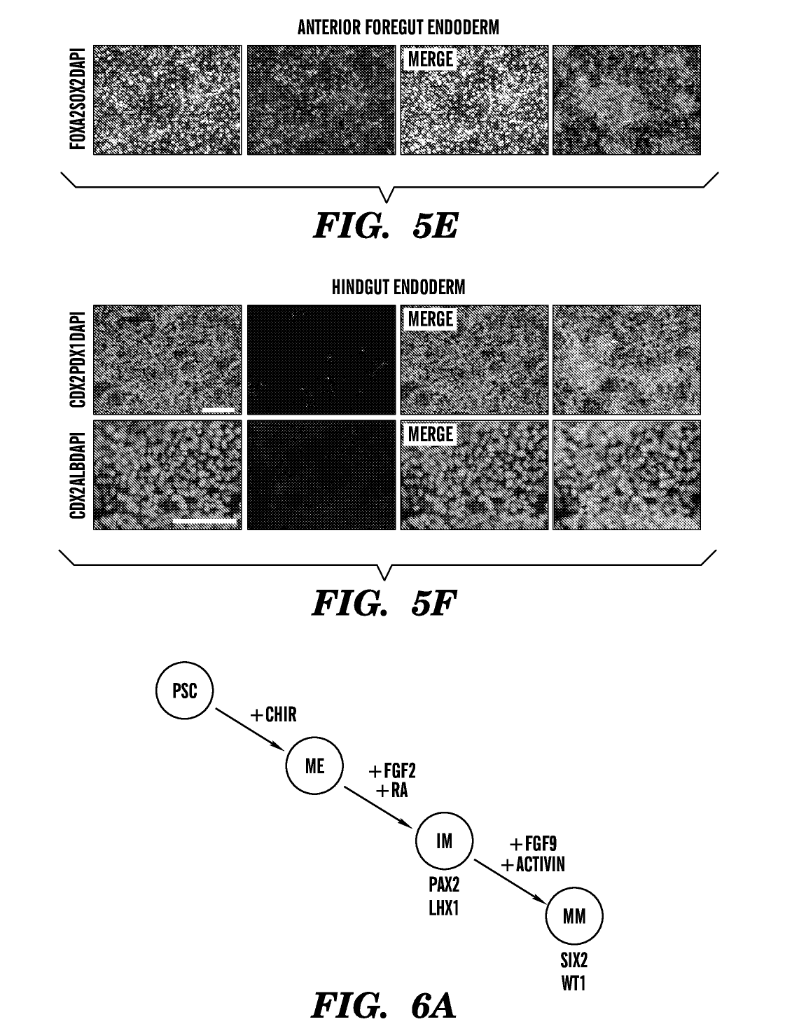

To determine whether CHIR-induced cells can be diverted from the default lateral plate mesoderm fate, the Inventors applied high doses of activin A to hPSCs after 24 hours of CHIR treatment, resulting in successful generation of SOX17+FOXA2+ definitive endoderm with greater than 95% efficiency after 2-3 days of subsequent differentiation (FIGS. 2F and 5). These definitive endoderm cells could be differentiated further into pancreatic endocrine cells expressing insulin and somatostatin (FIG. 2G), mature hepatocytes expressing albumin, anterior foregut endoderm (precursors to lung and thyroid tissue), and hindgut endoderm (precursors to intestine) (FIG. 5D-F). However, addition of activin A after 48 hours of CHIR treatment markedly reduced the number of SOX17+ cells after 4 days of differentiation (FIG. 5B), indicating that there was a distinct window of time during which CHIR-induced cells could be differentiated into different mesendodermal lineages. Collectively, these results highlighted the critical importance of timing and duration of signaling factors with regards to cell fate determination in the described differentiation system.

### Example 10

### FGF2 Induces PAX2 Expression in CHIR-Induced Mesendodermal Cells

Having demonstrated that one can manipulate the fate of CHIR-treated cells with the time-sensitive addition of specific inducing factors, the Inventors screened candidate growth factors for the ability to induce the expression of the intermediate mesoderm (“IM”) marker PAX2. PAX2 is selected since it is an early marker of IM, and, unlike the markers OSR1 or LHX1 which are also expressed in the adjacent lateral plate mesoderm, PAX2 expression is restricted in mesoderm to the IM. Human PSCs were induced with CHIR for 24 hours, at which time CHIR was withdrawn and cells were treated with increasing doses of activin A, BMP-2, BMP-4, BMP-7, FGF2, or retinoic acid (“RA”). On day 4 of differentiation, modest increases in PAX2 expression, compared to a vehicle control, were seen in cells treated with low doses of BMP-2, BMP-7, and RA, and no PAX2 expression was seen in cells treated with either activin or BMP-4 (FIG. 2I).

It is observed, however, that approximately 50-60% of cells treated with FGF2 at a dose of 100 ng/mL are positive for PAX2 by immunostaining, suggesting that FGF2 can be a potential inducer of IM. The ability of FGF2 to induce PAX2 expression was dependent upon cells being pre-treated with CHIR, as the addition of FGF2 to hPSCs not initially induced with CHIR resulted in the absence of PAX2 expression on day 4 of differentiation (FIG. 2J).

### Example 11

### FGF2 and Retinoic Acid Induce PAX2+LHX1+Intermediate Mesoderm Cells

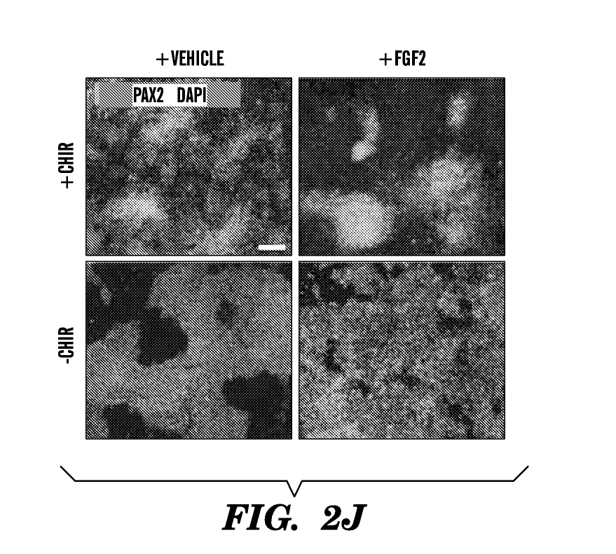

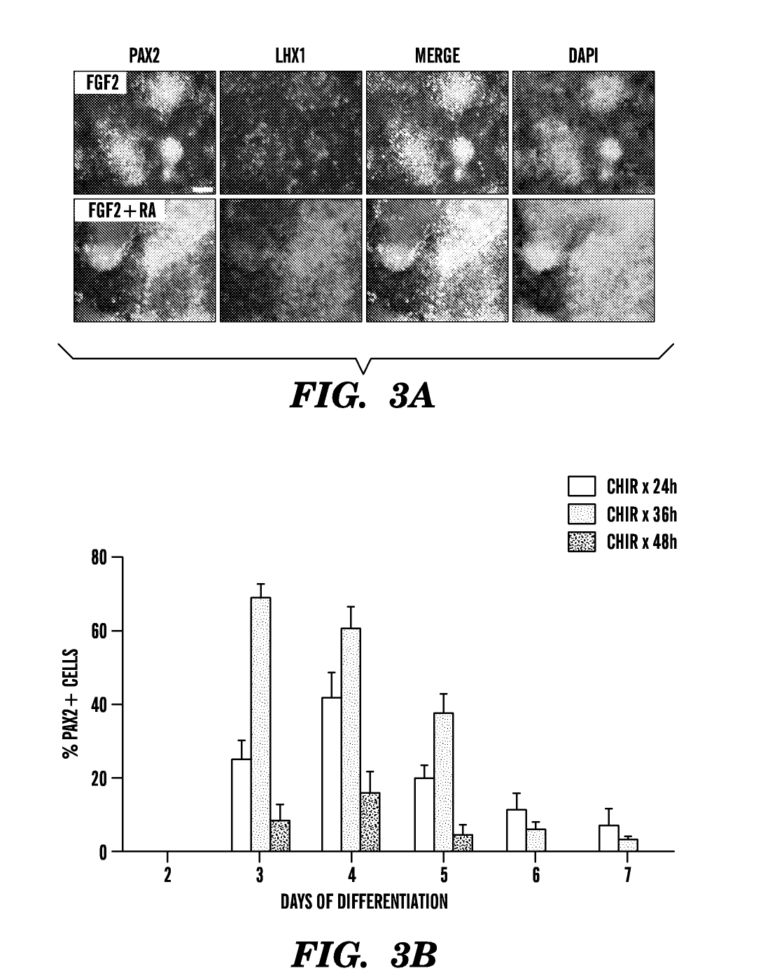

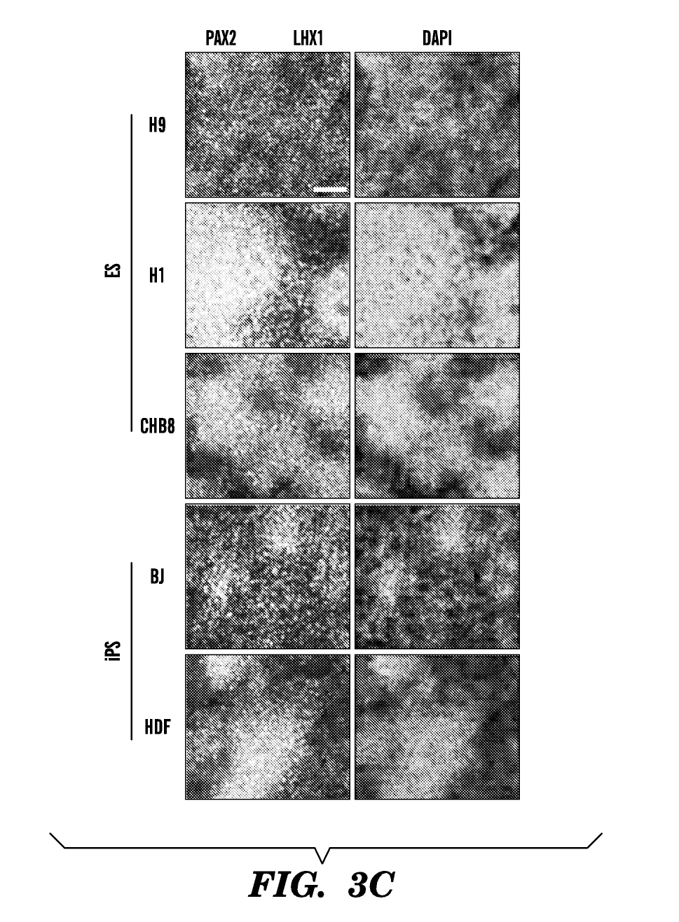

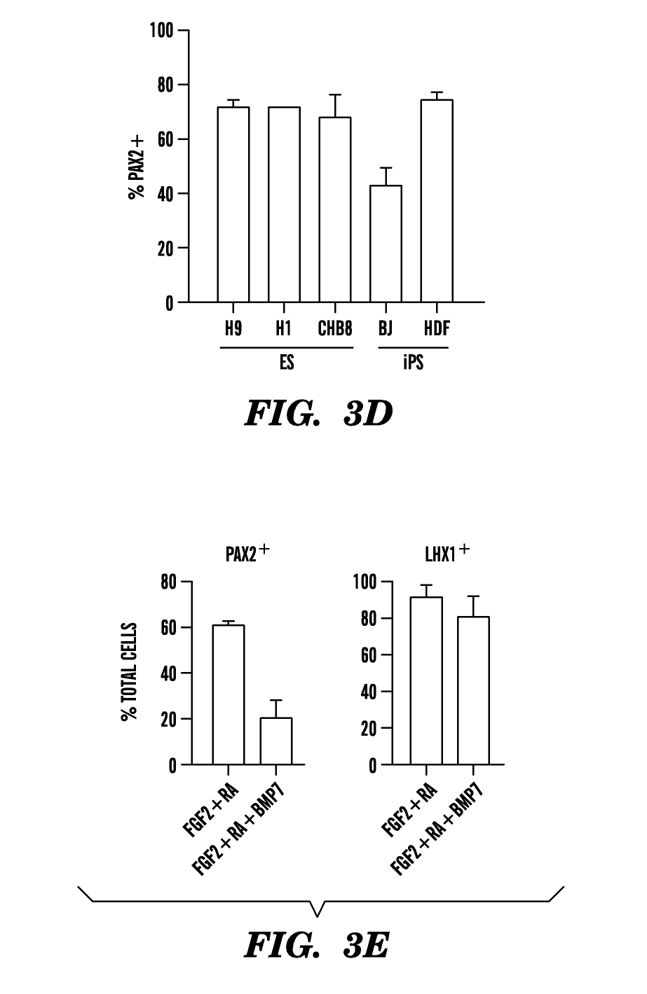

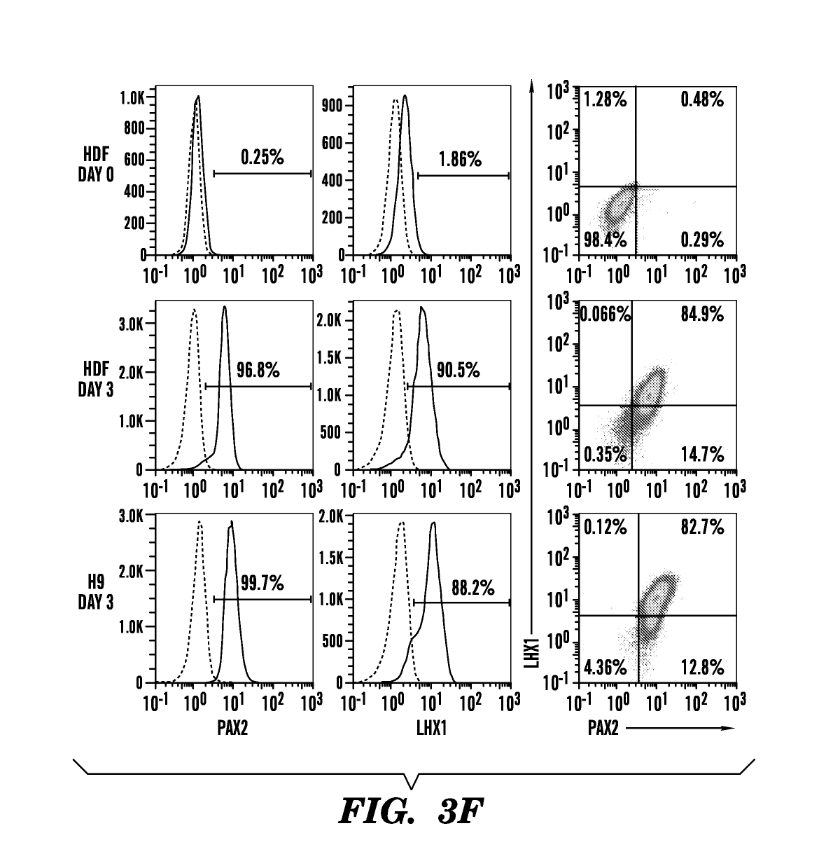

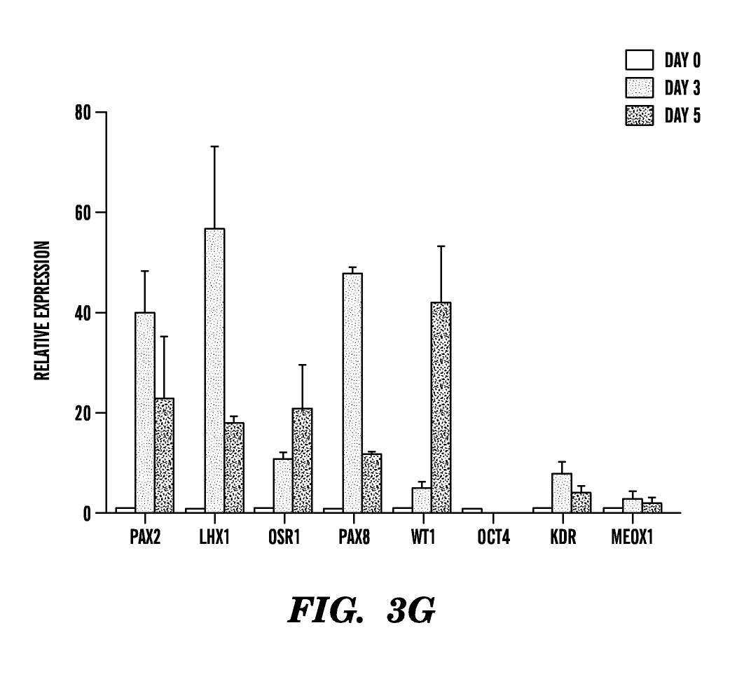

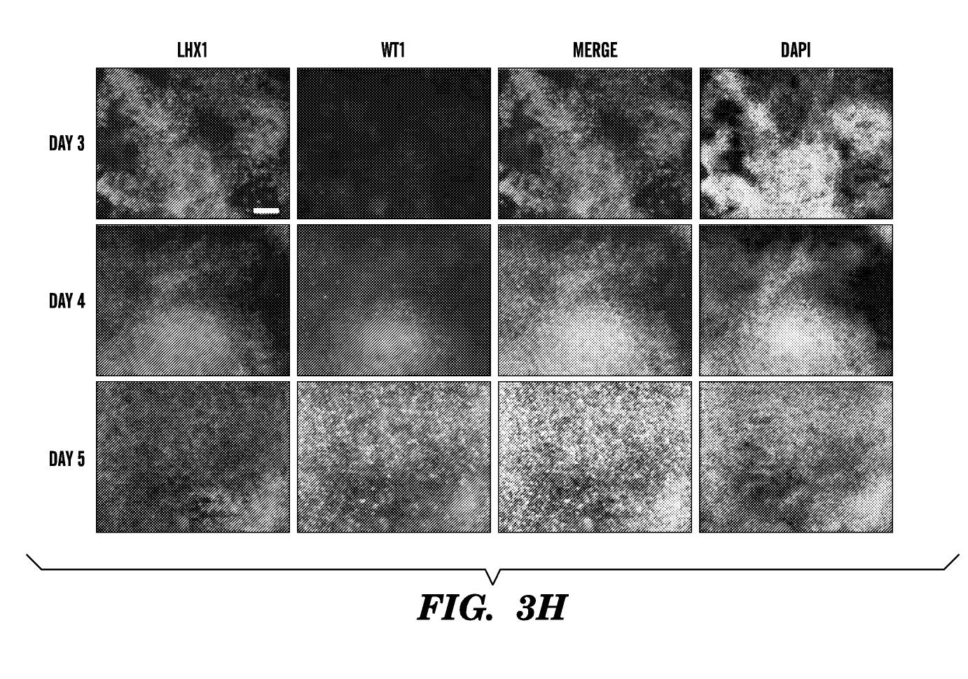

Retinoic acid signaling has previously been demonstrated to play an important role in the early stages of kidney development. Because a small inductive effect on PAX2 expression with RA is observed, low dose RA were tested in combination with FGF2 to determine whether it could have a synergistic effect on PAX2 expression. While this does not result in a dramatic increase in PAX2 expression, the addition of 1 μM RA resulted in a marked increase in LHX1 expression, with the majority of cells co-expressing both PAX2 and LHX1 (FIG. 3A).

As PAX2 and LHX1 are both expressed in the developing IM, these cells are labeled as a putative IM cell population. The Inventors sought to optimize the efficiency of generating IM cells. After testing the effects of different durations of CHIR pre-treatment and assaying PAX2 expression from days 2 through 7 of differentiation, it was observed that induction of hPSCs with CHIR for 36 hours followed by FGF2 and RA resulted in PAX2 expression in greater than 70% of cells as early as day 3 of differentiation (FIG. 3B). Longer pre-treatment with CHIR for 48 hours resulted in less PAX2 expression at all time points when compared to CHIR treatment for 24 or 36 hours. Regardless of the duration of CHIR pretreatment, the proportion of cells expressing PAX2 significantly decreased after day 4 of differentiation, with fewer than 10% of cells at day 7 retaining PAX2 expression. Since previous studies have identified a role for BMP-7 in inducing IM cells, the Inventors determined whether the addition of BMP-7 to FGF2 and RA could enhance IM cell differentiation. In contrast to these other reports, the Inventors found that the addition of BMP-7 significantly decreased the percentage of cells expressing PAX2 but had a minimal effect on the expression of LHX1 (FIG. 3E).

To determine the reproducibility of the described protocol in different hPSC lines, the Inventors tested the combination of CHIR induction for 36 hours followed by the addition of FGF2 and RA (ChFR) in three hESC lines and two hiPSC lines. Similar patterns of co-staining for PAX2 and LHX1 are observed in all cell lines with nearly identical efficiencies of differentiation (70-80%) in four of the five cell lines and a slightly reduced differentiation efficiency in one iPS cell line (FIG. 3C,D). To confirm these findings, the Inventors used flow cytometry to quantify PAX2 and LHX1 expression in hESCs and hiPSCs treated with this protocol. Interestingly, even higher proportions of differentiated cells were positive for PAX2 or LHX1 by flow cytometry, and 80-85% of cells were double-positive for PAX2 and LHX1 (FIG. 3F).

The Inventors then performed gene expression profiling of the putative IM cells using quantitative RTPCR. Consistent with the described protein expression data, the Inventors observed a marked upregulation of IM genes, including PAX2, LHX1, OSR1, and PAX8, on day 3 of differentiation, followed by a reduction in gene expression at day 5 (FIG. 3G), suggesting that IM differentiation in hPSCs may be a transient state which can be rapidly induced but lasts only 2-3 days. The expression of lateral plate and paraxial mesoderm markers KDR and MEOX1, respectively, was not significantly upregulated by the described IM induction protocol, and, as expected with differentiation, the Inventors noted downregulation of the pluripotency marker OCT4. Expression of WT1, a marker expressed first in the IM and then in the urogenital ridge which is derived from IM, was expressed at a low level on day 3 and was strongly upregulated on day 5 of differentiation (FIG. 3G,H). Since PAX2 and LHX1 are also expressed in the developing ear, eye, and central nervous system during embryogenesis, the Inventors evaluated the putative IM cells for the expression of markers (EYA1, EYA2, SOX3, OTX2, FOXI3, SIX4) which are co-expressed with PAX2 and/or LHX1 at relevant stages of neuroectodermal development and found that their expression was either downregulated or unchanged compared to undifferentiated hPSCs (FIG. 3I). Thus, PAX2+LHX1+ cell population is representative of IM.

### Example 12

### Human PSC-Derived Intermediate Mesoderm Cells Form Tubules which Express Proximal Tubular Markers

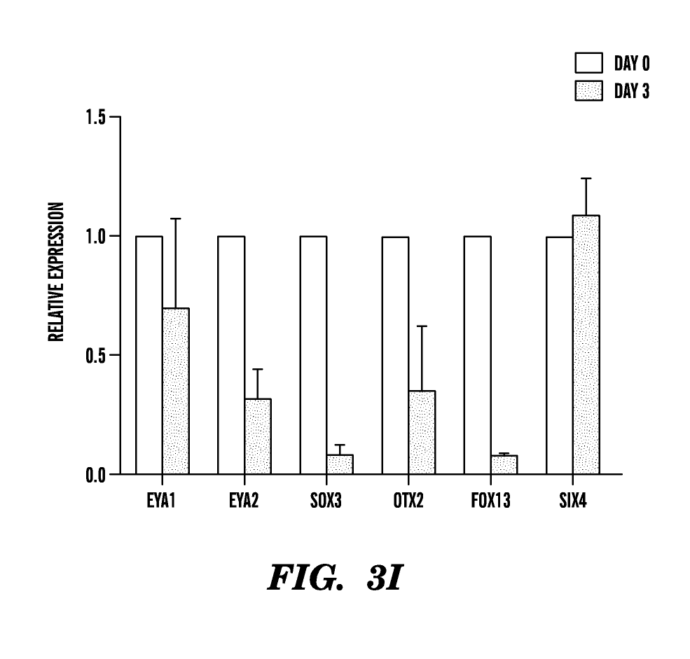

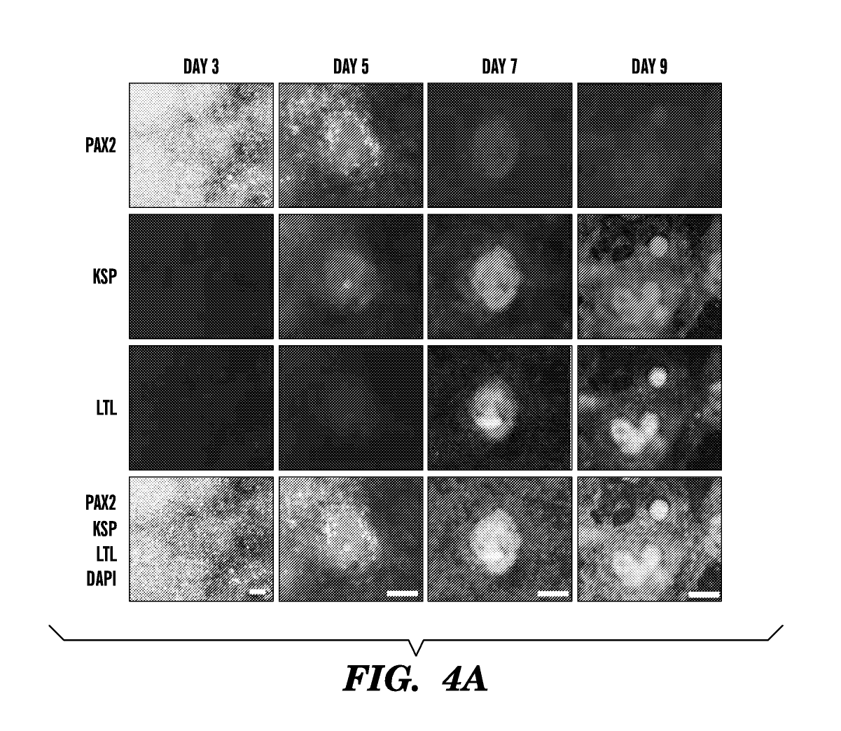

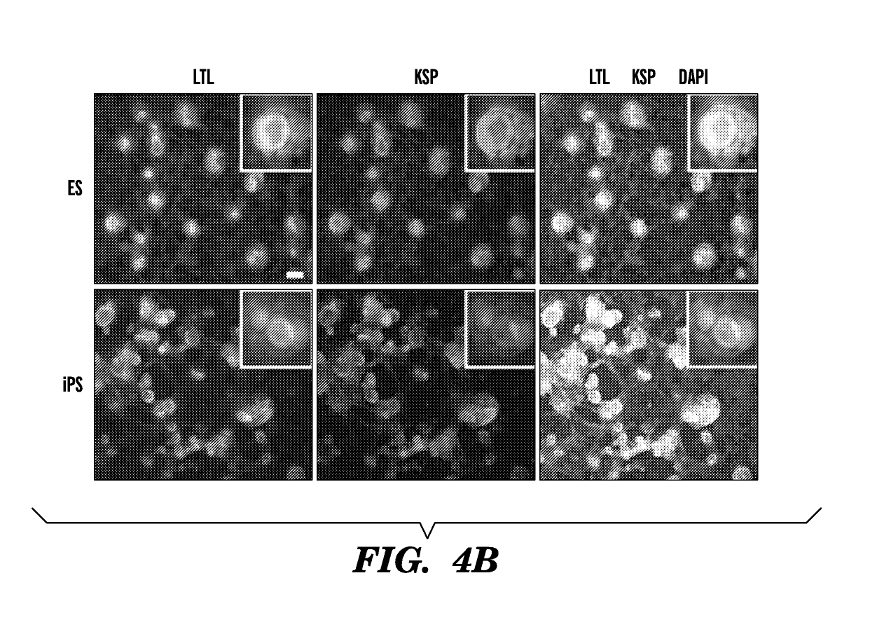

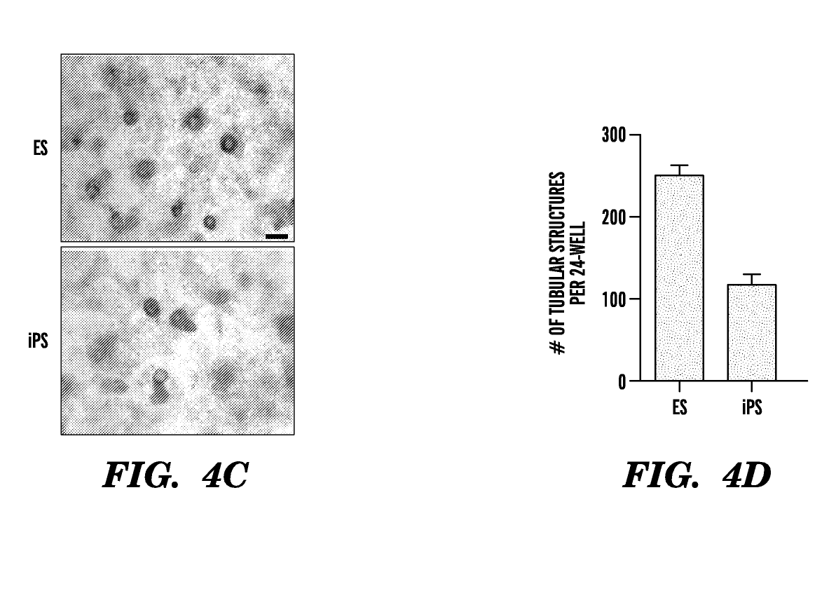

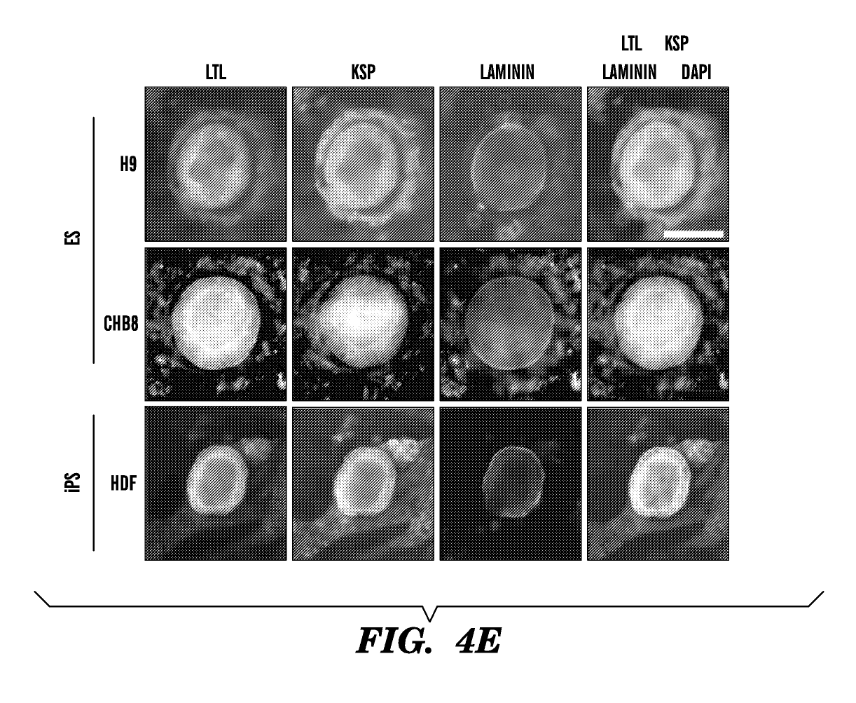

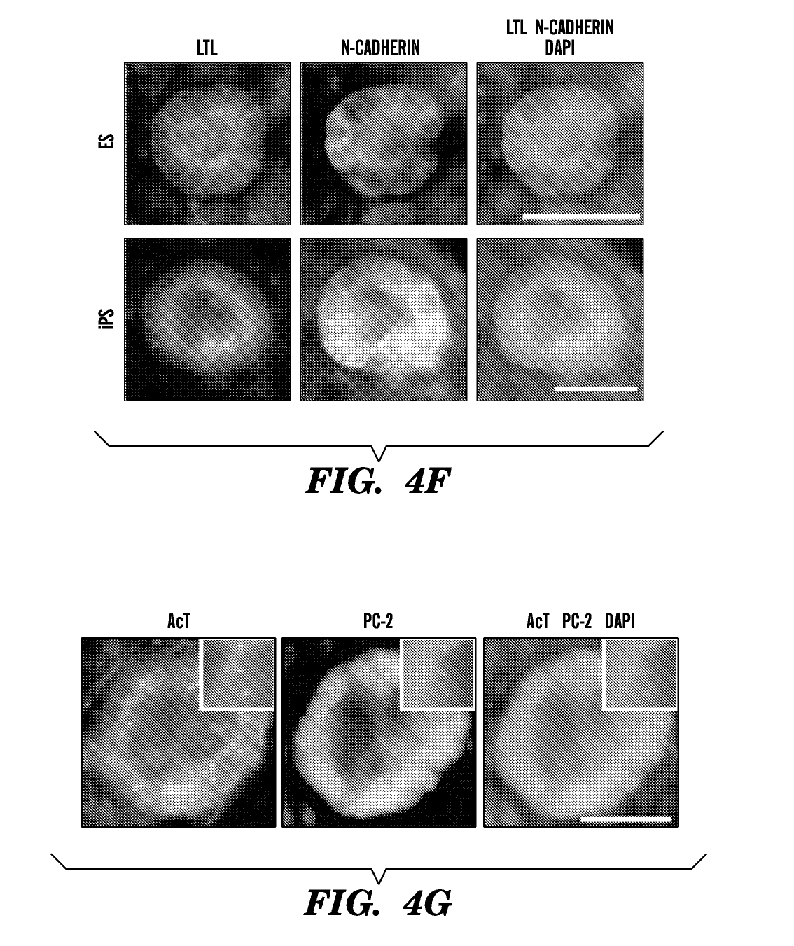

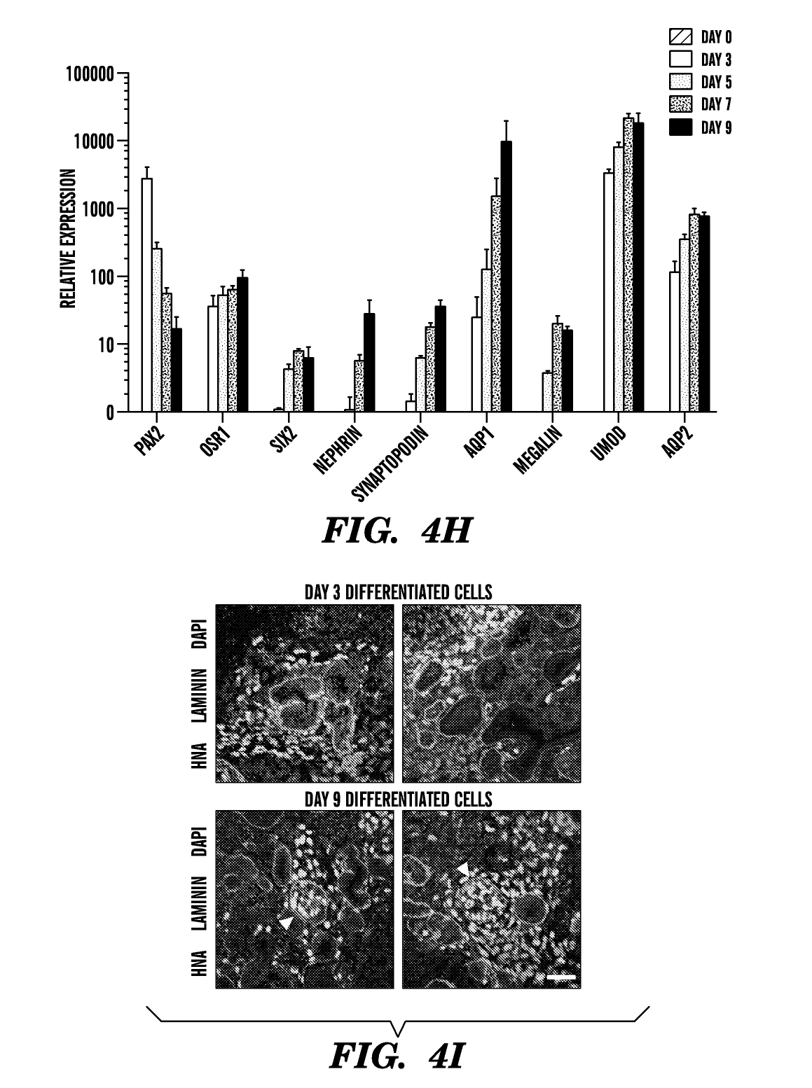

To determine whether hPSC-derived PAX2+LHX1+IM cells have the capacity to give rise to more differentiated cell and tissue derivatives of IM, the Inventors withdrew FGF2 and RA from the culture media on day 3 of differentiation and cultured themin serum-free media, containing no additional growth factors or chemicals, for another week. Cell growth and proliferation continued under these conditions, and as early as day 7, tubular epithelial structures formed in parallel with the downregulation of PAX2 expression (FIG. 4, A-D). Immunostaining for markers of more differentiated kidney cell types revealed that the cells comprising these tubular structures expressed the following: Lotus tetragonolobus lectin (LTL), which localizes to the apical surface of kidney proximal tubules; N-cadherin, which is the predominant cadherin expressed on proximal tubular cells; and kidney-specific protein (KSP), a cadherin that is known to be expressed on all kidney tubular epithelial cells andmarks mouse embryonic stem cell-derived kidney tubular cells, (FIGS. 4, E and F). The formation of laminin-bounded tubular structures coexpressing LTL, KSP, and N-cadherin was reproducible in structures derived from both hESC and hiPSC lines (FIGS. 4, E and F). Furthermore, the Inventors observed the presence of primary cilia expressing the ciliary protein polycystin-2 on the apical surface of many of the tubular structures (FIG. 4G), another feature of polarized kidney tubules that confirms the polarity of the epithelial cells. The Inventors then evaluated the expression of more differentiated kidney markers in cells treated with the IM-inducing protocol from days 0 to 9 and observed a significant, time-dependent upregulation in the expression of SIX2, a marker for multipotent nephron progenitor cells of the metanephric mesenchyme, and markers of mature kidney epithelial cells, such as NEPHRIN (podocyte), SYNAPTOPODIN (podocyte), AQP1 (proximal tubule), MEGALIN (proximal tubule), UMOD (loop of Henle), and AQP2 (collecting duct) (FIG. 4H).

### Example 13

### Chimeric Kidney Explant Cultures: Re-Aggregation Assay

To further confirm the identity of these cells as embryonic kidney cells, the Inventors subjected the cells to kidney explant re-aggregation assays. Chimeric kidney explants can be created using well-known re-aggregation techniques. Briefly, embryonic kidneys at stage E12.5 (day of plug=E0.5) are isolated from timed pregnant females (Charles River). Complete urogenital systems are placed in DMEM (Corning Cellgro) and further dissected to isolate single E12.5 kidneys. 4-6 kidneys were incubated in TrypLE™ Express (Invitrogen) at 37° C. for 4 minutes. The enzyme is then quenched by adding Kidney Culture Media (KCM: DMEM+1×P/S+10% fetal bovine serum) and incubating at 37° C. for 10 minutes for recovery. Digested kidney rudiments were then transferred to a microcentrifuge tube with additional KCM and dissociated by repeated trituration. The cell suspension is then passed through a 100 μm pore size cell strainer before visualizing toconfirm single cell suspension and counting. Differentiated human pluripotent stem cells from day 3 and day 9 are dissociated with TrypLE, visualized, and counted. Re-aggregation is done by mixing 130,000 dissociated mouse kidney cells with 13,000 differentiated human cells in a microcentrifuge tube and centrifuging the chimeric mixture into a pellet at 700×g. The resulting chimeric pellet are then placed onto a Nucleopore Track-Etch Membrane filter disk (pore size=1 μm) (Whatman) 60, and the filter floated on 1 ml KCM+10 μM Y27632 61 in a 24-well tissue culture plate (two explants per 13 mm circular filter) and incubated for 24 hours. After the initial incubation, the media with Y27632 is replaced with KCM only and cultured for an additional 2 days.

### Example 14

### Chimeric Kidney Explant Cultures

IM cells are dissociated on days 3 (PAX2+LHX1+) or 9 (LTL+KSP+) of differentiation and recombined with dissociated cells from wild-type E12.5 mouse embryonic kidneys.

Human cells from day 3 can incorporate into mouse metanephric tissues, distributing in the interstitium; however, no tubular integration was observed. Human cells from day 9 are found not only in the mouse metanephric interstitium but are also identified within organized laminin-bounded structures which also contained mouse cells (FIG. 4H). These structures were similar in morphology to other laminin-bounded structures in the co-culture reaggregate which contained only mouse metanephric cells. The Inventors therefore concluded that the ability of hPSC-derived PAX2+LHX1+IM cells to form tubular structures in vitro and ex vivo is consistent with their identification as embryonic kidney cells.

### Example 15

### Generation of Metanephric Mesenchyme

Following generation of intermediate mesoderm cells, the Inventors have further established a method to differentiate the intermediate mesoderm cells further into cells of the metanephric mesenchyme, which express the markers SIX2 and WT1. This population of expressing SIX2 and WT1 gives rise to most of the epithelial cells in the kidney, and further validates the identity of generated cells as primitive intermediate mesoderm cells.

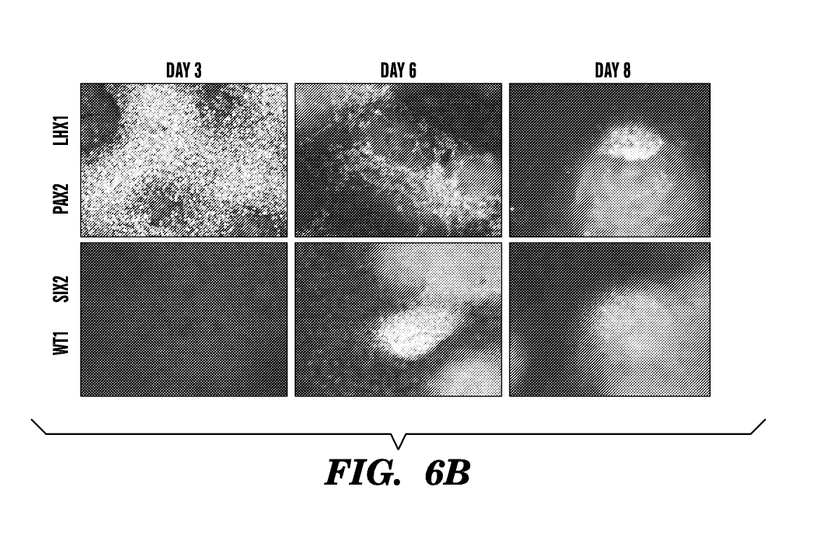

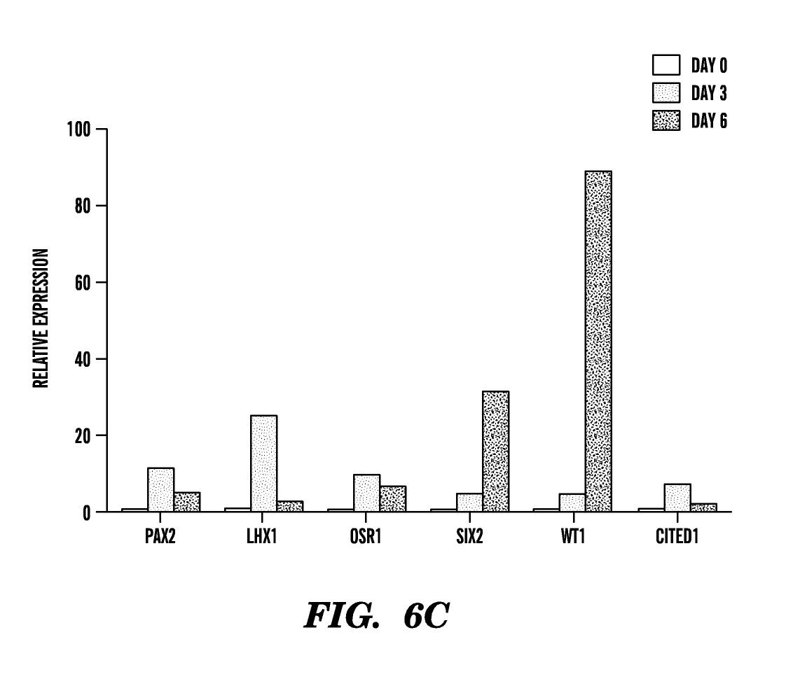

As shown in FIG. 6, PAX2+LHX1+ intermediate mesoderm cells can be differentiated further into SIX2+WT1+ of the metanephric mesenchyme, and various types of mesendoderm, intermediate mesoderm, and metanephric mesenchyme are shown (FIG. 6A). Immunocytochemistry for PAX2, LHX1, WT1, and SIX2 in hPSCs treated with CHIR for 36 hours, then FGF2 100 ng/mL+RA 1 μm for 42 hours demonstrate the appearance of intermediate mesoderm cells, with further culturing in the presence of FGF9 100 ng/ml+Activin A 10 ng/mL, at Days 3, 6, and 9 demonstrating the generation of metanephric mesenchyme. It is noted that the markers for this lineage, SIX2 and WT1 co-expression is observed as early as day 6 of differentiation (FIG. 6B). These results are confirmed using quantitative RT-PCR of genes expressed in the intermediate mesoderm and metanephric mesenchyme in hPSCs treated with CHIR for 36 hours, FGF2+RA for 42 hours, then FGF9 and activin A. SIX2 and WT1 expression are highly upregulated on Day 6 compared to Day 0 and Day 3 (FIG. 6C).

### Example 16

### FGF9 and Activin A Induce Expression of CM Markers in PAX2+LHX1+Cells

During embryonic kidney development, the CM comprises a population of multipotent nephron progenitor stem cells that express the transcription factor SIX2 and give rise to nearly all the epithelial cells of the nephron, with the exception of the collecting duct cells. Although SIX2 mRNA levels increased during stochastic differentiation into tubular structures (FIG. 4H), SIX2 protein was not clearly detectable by immunofluorescence, suggesting that its stable expression may require additional factors.

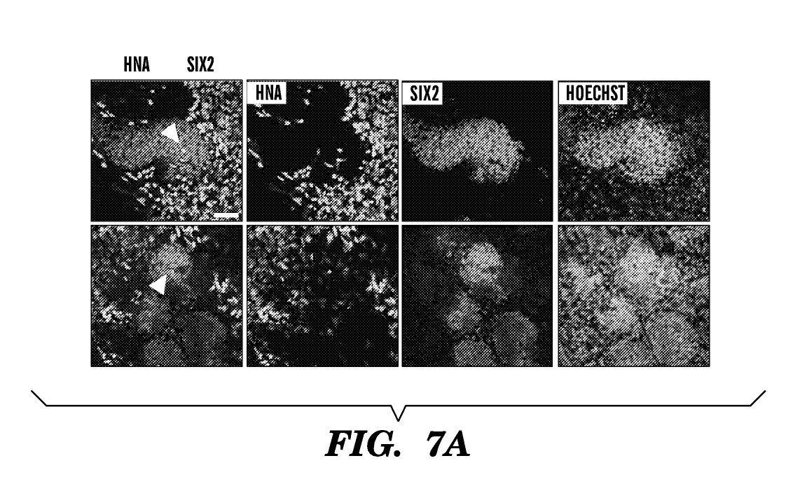

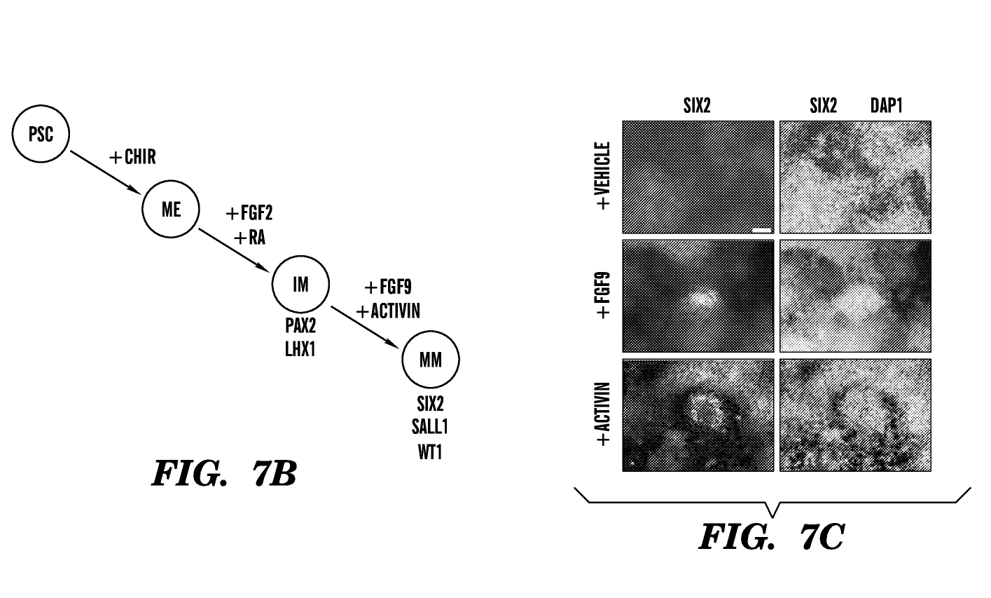

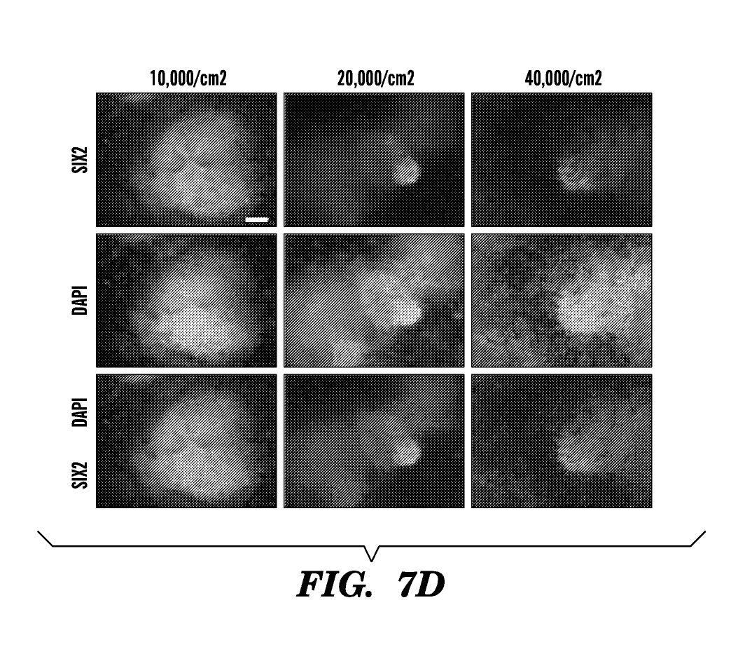

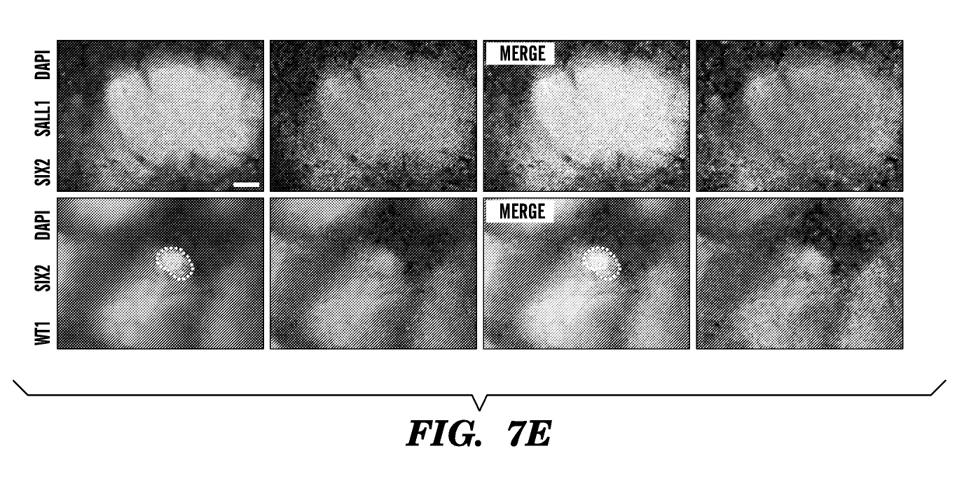

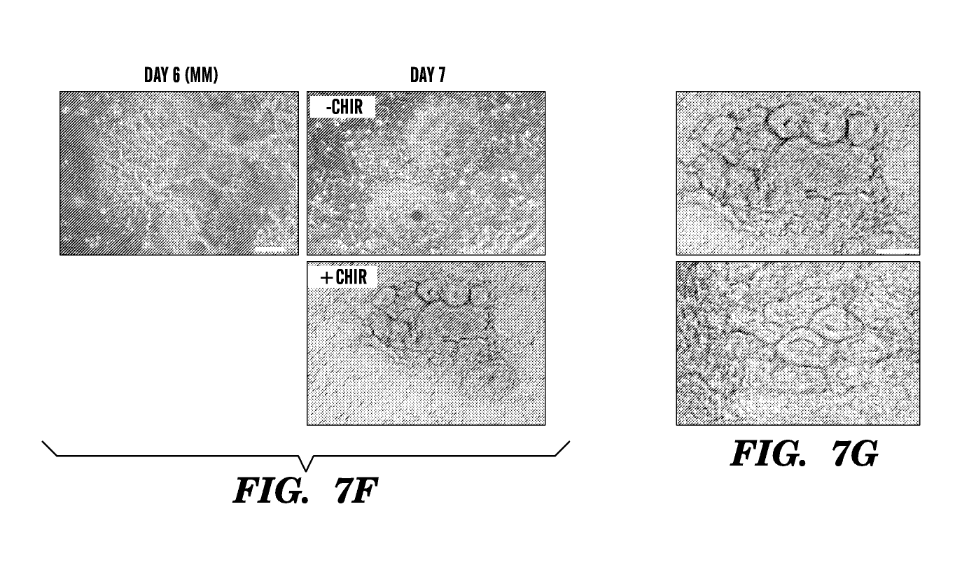

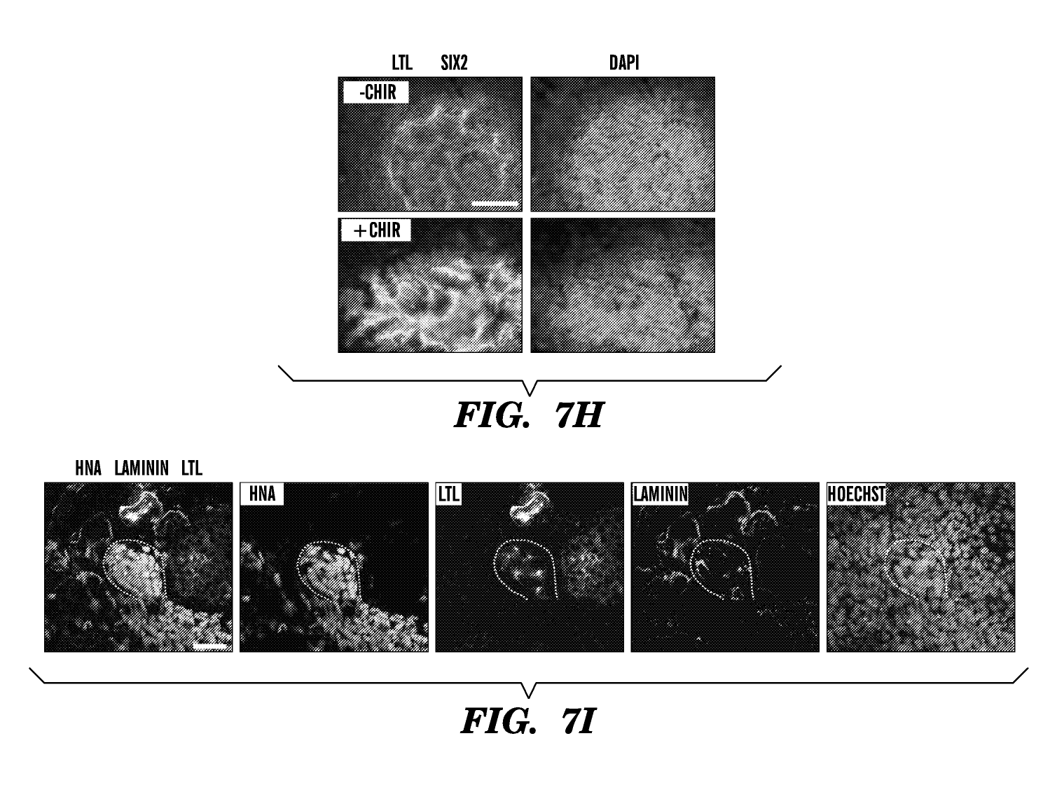

In addition to forming chimeric laminin-bounded structures in recombination explant culture (FIG. 4I), stochastically differentiated human PAX2+LHX1+ cells incorporated into clusters of Six2+ mouse metanephric cells but did not express SIX2 protein by immunofluorescence (FIG. 7A). To identify conditions that promote and sustain a SIX2+ cell population in vitro, the Inventors screened growth factors added on day 3 of differentiation for the ability to induce SIX2 expression detectable by immunofluorescence (FIG. 7B). From our initial screen, the Inventors observed small populations of SIX2+ cells when PAX2+LHX1+ cells were treated with FGF9 100 ng/ml or activin A 10 ng/ml for 5 additional days, whereas no SIX2+ cells were seen with treatment with a vehicle control (FIG. 7C). The combination of FGF9 and activin A, together with decreasing the initial cell plating density, markedly improved the efficiency of SIX2 induction and demonstrated that SIX2 expression could be seen as early as day 6 of differentiation (FIG. 7D).

To confirm that this SIX2 expression was consistent with differentiation toward CM, the Inventors evaluated the expression of SALL1 and WT1, two other important markers of CM.34,45 Nearly all SIX2+ cells coexpressed SALL1 as seen by immunocytochemistry, and a subset of SIX2+ cells also coexpressed WT1 (FIG. 7E). During embryonic kidney development, Wnt signals from the ureteric bud induce the CM to condense into a pretubular aggregate, which subsequently develops into a renal vesicle, comma-shaped body, S-shaped body, and ultimately a nephron. 20 In vitro, FACS isolated mouse Six2+ cells transiently induced by Wnt signaling (using the GSK-3b inhibitor BIO) undergo epithelialization and begin expressing markers of nephron development.

To test the competence of hPSC-derived SIX2+ cells to respond to canonical Wnt signaling, the Inventors treated cells on day 6 of differentiation with 5 mM CHIR for 24 hours, followed by withdrawal of CHIR. Within 24 hours of CHIR treatment, the Inventors observed distinct changes in cell morphology and the formation of tubular-like structures (FIGS. 7, F and G). Immunocytochemistry of these structures on day 8 of differentiation revealed a downregulation of SIX2 expression and increased expression of the proximal tubule marker LTL in CHIR-induced cells compared with cells not treated with CHIR (FIG. 7H), suggesting that treatment with CHIR had induced changes similar to that seen with induction of CMand the initiation of tubulogenesis in vivo. Furthermore, hPSC-derived SIX2+ cells explanted into dissociated-reaggregated mouse embryonic kidneys formed organizing clusters of cells that expressed LTL (FIG. 7I). The Inventors therefore concluded that the ability of hPSCderived PAX2+LHX1+ cells to be differentiated into cells expressing multiple markers of kidney CMand that could form tubule like structures in response to Wnt signaling was consistent with the behavior and function of nephrogenic IM cells.

### Example 17

### Summary of Results

Described herein is a rapid, efficient, and highly reproducible system to induce intermediate mesoderm cells from hESCs and hiPSCs under precise, chemically defined, monolayer culture conditions. Robust generation of a BRACHYURY+MIXL1+ mesendodermal cell population with the use of CHIR99021 confirmed the potency of GSK-3β inhibitors to generate mesendoderm and established the proper platform for us to screen compounds which could effectively promote IM differentiation.

By investigating the differentiation kinetics of CHIR-treated hPSCs, the Inventors established that increasing exposure to CHIR resulted in differentiation towards a lateral plate mesoderm fate, but this default pathway could be altered by the precisely-timed addition of fate altering growth factors. This affirms the findings of prior reports demonstrating that mesodermal and endodermal cell fates are determined by a delicate time- and dose-dependent balance of Wnt, activin, BMP, and FGF signaling. These findings, however, contrast with those of a recent study by Tan and colleagues, in which the authors showed that prolonged exposure to GSK-3β inhibition, specifically with CHIR, promoted an endodermal rather than mesodermal fate. With the described differentiation conditions, definitive endoderm differentiation could only be achieved with the synergistic effect of CHIR and high-dose activin.

While the differentiation of hPSCs into cells of the cardiac, hepatic, pancreatic, and neuronal lineages have been widely reported, previous attempts to derive cells of the kidney lineage from hPSCs have been relatively few in number. An alternative to directed differentiation was recently demonstrated by means of direct reprogramming of immortalized human kidney cells into nephron progenitor-like cells; however, the efficiency of integration into kidney explant cultures was reportedly low. Others have reported induction of an intermediate mesoderm population using a stepwise combination of CHIR, activin, and BMP-7 signaling in engineered OSR1-GFP hiPSC cell lines, achieving efficiencies of greater than 90% of OSR1-GFP+ cells after 11-18 days of differentiation. However, because OSR1 is expressed in both the lateral plate and intermediate mesoderm during early mesoderm specification, this expression pattern does not distinguish intermediate from lateral plate mesoderm, and the proportion of OSR1+ cells which co-expressed other important IM markers such as PAX2 or WT1 was comparatively low.

The Inventors selected PAX2 and LHX1 as more specific markers of IM for the purpose of defining IM inducing culture conditions. It is important to note that the expression of PAX2 and LHX1 is not limited to the developing kidney during embryogenesis and can be seen at other stages of development in the eye, ear, and central nervous system; however, co-expression of PAX2 and LHX1 within the same domain has only been described in the developing kidney and dorsal spinal cord. Importantly, the above described result identify, for the first time, that FGF2 is a potent factor in inducing PAX2 expression in CHIR-induced mesendodermal cells. When combined with RA, this combination is able to robustly generate a PAX2+LHX1+IM cell population as confirmed by both immunocytochemistry and flow cytometry.

This described protocol is capable of achieving efficient IM differentiation within 3 days, which is considerably quicker than existing protocols while maintaining a high level of efficiency, and was highly reproducible in multiple hESC and hiPSC lines without the need for flow sorting. Interestingly, with the described culture conditions it was observed that the addition of BMP-7, which has been used as a component of other kidney-lineage differentiation protocols, did not have an synergistic effect in inducing IM differentiation. While the precise conditions for specifically generating other IM derivatives, including the adrenal cortices and gonads, have yet to be defined, the Inventors demonstrated that inducing PAX2+LHX1+ cells is sufficient for these cells to autonomously express WT1, a later marker of IM differentiation, and to form polarized, ciliated tubular structures which express markers of kidney proximal tubular cells and integrate into mouse metanephric cultures. These polarized tubular structures could reproducibly form in monolayer culture, in contrast to previous reports in which tubular structures derived from differentiated hPSCs cells could form only with 3D culture in vitro or after incorporation into mouse metanephric kidneys ex vivo.

Described herein is the first report of the generation of SIX2+ cells from hPSCs. The described method of using FGF9 to induce SIX2 expression is consistent with the important role of FGF9 in maintaining the nephron progenitor population during embryonic kidney development. As described, when SIX2+ cells were transplanted ex vivo into mouse metanephric cultures, they organized into structures that expressed LTL and laminin. In parallel, activation of canonical Wnt signaling in the SIX2+ cell population using CHIR resulted in the rapid formation of tubule-like structures in vitro in which cells downregulated SIX2 and expressed LTL. Although this result suggests that hPSC-derived SIX2+ cells can be induced to condense and epithelialize in a manner similar to that seen with CMin vivo, further studies are needed to determine the precise conditions for activating a program of kidney tubulogenesis.

In conclusion, it is demonstrated herein that sequential treatment with CHIR99021 and FGF2 and RA induces efficient differentiation of hPSCs into PAX2+LHX1+ intermediate mesoderm, and that these cells are capable of giving rise to polar ciliated tubular structures which express markers of kidney proximal tubular epithelial cells. The establishment of this system will facilitate and improve the directed differentiation of hPSCs into cells of the kidney lineage for the purposes of bioengineering kidney tissue and iPS cell disease modeling.

As described herein, the Inventors have established is a highly efficient system to differentiate hESCs and hiPSCs into cells of the intermediate mesoderm, these cells being capable of expressing IM-specific markers, PAX2+LHX1+, autonomous WT1 expression, in addition to formation of tubules expressing differentiated kidney markers such as LTL, cilia with polycystin-2 protein and integration into mouse embryonic kidney explant cultures Treatment of hPSCs with the GSK-3β inhibitor CHIR99021 induced BRACHYURY+MIXL1+ mesendoderm differentiate with nearly 100% efficiency. Whereas the absence of additional exogenous factors leads CHIR-induced mesendodermal cells to preferentially differentiate into lateral plate mesoderm with minimal IM differentiation, the sequential treatment of hPSCs with CHIR followed by FGF2 and retinoic acid generated PAX2+LHX1+ cells with remarkable speed, 3 days, and at high efficiency of up to 70-80%. The described protocols establish for the first time the effective role of FGF signaling in inducing IM differentiation in hPSCs and establish the most rapid and efficient system whereby hPSCs can be robustly differentiated into kidney tubulogenic PAX2+LHX1+IM cells. The addition of FGF-9 and activin more specifically differentiates PAX2+LHX1+ cells into cells expressing SIX2, SALL1, and WT1, markers of the nephron progenitor stemcell pool in the CM, further demonstrating that PAX2+LHX1+ cells have the potential to give rise to IM derivatives. The establishment of this system will facilitate and improve the directed differentiated of hPSCs into cells of the kidney lineage for the purposes of bioengineering kidney tissue and iPS cell disease modeling.

The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as may be taught or suggested herein. A variety of advantageous and disadvantageous alternatives are mentioned herein. It is to be understood that some preferred embodiments specifically include one, another, or several advantageous features, while others specifically exclude one, another, or several disadvantageous features, while still others specifically mitigate a present disadvantageous feature by inclusion of one, another, or several advantageous features.

Furthermore, the skilled artisan will recognize the applicability of various features from different embodiments. Similarly, the various elements, features and steps discussed above, as well as other known equivalents for each such element, feature or step, can be mixed and matched by one of ordinary skill in this art to perform methods in accordance with principles described herein. Among the various elements, features, and steps some will be specifically included and others specifically excluded in diverse embodiments.

Although the invention has been disclosed in the context of certain embodiments and examples, it will be understood by those skilled in the art that the embodiments of the invention extend beyond the specifically disclosed embodiments to other alternative embodiments and/or uses and modifications and equivalents thereof.
Many variations and alternative elements have been disclosed in embodiments of the present invention. Still further variations and alternate elements will be apparent to one of skill in the art. Among these variations, without limitation, are compositions for generating intermediate mesoderm, methods of generating intermediate mesoderm, cells and cell lines produced by the described methods and compositions, including undifferentiated cells and their differentiated progeny, and the particular use of the products created through the teachings of the invention. Various embodiments of the invention can specifically include or exclude any of these variations or elements.

In some embodiments, the numbers expressing quantities of ingredients, properties such as concentration, reaction conditions, and so forth, used to describe and claim certain embodiments of the invention are to be understood as being modified in some instances by the term “about.” Accordingly, in some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the invention are approximations, the numerical values set forth in the specific examples are reported as precisely as practicable. The numerical values presented in some embodiments of the invention may contain certain errors necessarily resulting from the standard deviation found in their respective testing measurements.

In some embodiments, the terms “a” and “an” and “the” and similar references used in the context of describing a particular embodiment of the invention (especially in the context of certain of the following claims) can be construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Preferred embodiments of this invention are described herein, including the best mode known to the inventors for carrying out the invention. Variations on those preferred embodiments will become apparent to those of ordinary skill in the art upon reading the foregoing description. It is contemplated that skilled artisans can employ such variations as appropriate, and the invention can be practiced otherwise than specifically described herein. Accordingly, many embodiments of this invention include all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above cited references and printed publications are herein individually incorporated by reference in their entirety.

In closing, it is to be understood that the embodiments of the invention disclosed herein are illustrative of the principles of the present invention. Other modifications that can be employed can be within the scope of the invention. Thus, by way of example, but not of limitation, alternative configurations of the present invention can be utilized in accordance with the teachings herein. Accordingly, embodiments of the present invention are not limited to that precisely as shown and described.

1. A method for generating a mesoderm cell, comprising:
providing a quantity of human pluripotent stem cells (“hPSCs”); and
culturing the hPSCs in a serum-free media comprising at least one induction molecule, wherein the at least one induction molecule is capable of generating at least one mesoderm cell.

2. The method of claim 1, wherein the human pluripotent stem cells are human embryonic stem cells (“hESCs”).

3. The method of claim 1, wherein the human pluripotent stem cells are human induced pluripotent stem cells (“hiPSCs”).

4. The method of claim 1, wherein the at least mesoderm cell is an intermediate mesoderm cell.

5. The method of claim 4, wherein the intermediate mesoderm cell expresses paired box-2 (“PAX2”), LIM homeobox-1 (“LHX”), and/or Wilms tumor-1 (“WT1”).

6. The method of claim 1, wherein the at least one induction molecule is a Glycogen synthase kinase-3 beta (“GSK3β”) inhibitor.

7. The method of claim 6, wherein the GSK3P inhibitor is CHIR99021.

8. The method of claim 7, wherein the hPSCs are cultured in a serum-free media comprising at least one induction molecule for about 12, 24, 36, or 48 hours.

9. The method of 1, comprising further culturing of the at least one mesoderm cell in the presence of at least one growth factor.

10. The method of claim 9, wherein the at least one growth factor comprises fibroblast growth factor-2 (“FGF2”) and/or retinoic acid (“RA”).

11. The method of claim 10, wherein further culturing of the at least one mesoderm cell in the presence of FGF2 and/or RA is for about 36, 48, 60, or 72 hours.

12. The method of claim 10, comprising further culturing in the presence of fibroblast growth factor-9 (“FGF9”) and/or activin A.

13. A composition of at least one mesoderm cell generated by the method of claim 1.

14. A pharmaceutical composition, comprising:
at least one mesoderm cell generated by the method of claim 1; and
a pharmaceutically acceptable carrier.

15. A composition of at least one mesenchyme cell generated by the method of claim 12.

16. An efficient method for generating intermediate mesoderm cells, comprising:
providing a quantity of human pluripotent stem cells (“hPSCs”);
culturing the hPSCs in a serum-free media comprising CHIR99021 for about 12, 24, 36, or 48 hours; and
further culturing in the presence of fibroblast growth factor-2 (“FGF2”) and/or retinoic acid (“RA”) for about 36, 48, 60, or 72 hours, wherein the culturing and further culturing generating intermediate mesoderm cells that express paired box-2 (“PAX2”) and LIM homeobox-1 (“LHX”).

17. The method of claim 16, comprising further culturing in the presence of fibroblast growth factor-9 (“FGF9”) and/or activin A.

18. The method of claim 16, wherein the method generates at least 50%, 60, 70% or more intermediate mesoderm cells.


SEQUENCE LISTING (68 sequences)

SEQ ID NO: 1 - DNA, 20 bp, Artificial Sequence
  AQP1 forward primer
attaaccctgctcggtcctt

SEQ ID NO: 2 - DNA, 20 bp, Artificial Sequence
  AQP1 reverse primer
accctggagttgatgtcgtc

SEQ ID NO: 3 - DNA, 20 bp, Artificial Sequence
  AQP2 forward primer
acgccttcacgtgtgtgtat

SEQ ID NO: 4 - DNA, 20 bp, Artificial Sequence
  AQP2 reverse primer
ttgttttctgcgccgaagtg

SEQ ID NO: 5 - DNA, 18 bp, Artificial Sequence
  B-ACTIN forward primer
ccaaccgcgagagagtga

SEQ ID NO: 6 - DNA, 18 bp, Artificial Sequence
  B-ACTIN reverse primer
tccatcacgatgccagtg

SEQ ID NO: 7 - DNA, 20 bp, Artificial Sequence
  BMP-4 forward primer
aagcgtagccctaagcatca

SEQ ID NO: 8 - DNA, 20 bp, Artificial Sequence
  BMP-4 reverse primer
tggttgagttgaggtggtca

SEQ ID NO: 9 - DNA, 26 bp, Artificial Sequence
  BRACHYURY forward primer
gtgctgtcccaggtggcttacagatg

SEQ ID NO: 10 - DNA, 27 bp, Artificial Sequence
  BRACHYURY reverse primer
ccttaacagctcaactctaactacttg

SEQ ID NO: 11 - DNA, 19 bp, Artificial Sequence
  CXCR4 forward primer
caccgcatctggagaacca

SEQ ID NO: 12 - DNA, 21 bp, Artificial Sequence
  CXCR4 reverse primer
gcccatttcctcggtgtagtt

SEQ ID NO: 13 - DNA, 22 bp, Artificial Sequence
  EOMES forward primer
atcattacgaaacagggcaggc

SEQ ID NO: 14 - DNA, 22 bp, Artificial Sequence
  EOMES reverse primer
cggggttggtatttgtgtaagg

SEQ ID NO: 15 - DNA, 20 bp, Artificial Sequence
  EYA1 forward primer
tgcatatgggcaaacacagt

SEQ ID NO: 16 - DNA, 20 bp, Artificial Sequence
  EYA1 reverse primer
ccaggttgaggggtactgaa

SEQ ID NO: 17 - DNA, 20 bp, Artificial Sequence
  EYA2 forward primer
ccggtctaagaggagcagtg

SEQ ID NO: 18 - DNA, 20 bp, Artificial Sequence
  EYA2 reverse primer
ctggtcacaatcctccaggt

SEQ ID NO: 19 - DNA, 24 bp, Artificial Sequence
  FOXA2 forward primer
ccattgctgttgttgcagggaagt

SEQ ID NO: 20 - DNA, 24 bp, Artificial Sequence
  FOXA2 reverse primer
caccgtgtcaggattgggaatgct

SEQ ID NO: 21 - DNA, 20 bp, Artificial Sequence
  FOXF1 forward primer
cagcctcacatcacgcaagg

SEQ ID NO: 22 - DNA, 19 bp, Artificial Sequence
  FOXF1 reverse primer
agccgagctgcaaggcatc

SEQ ID NO: 23 - DNA, 20 bp, Artificial Sequence
  FOXI3 forward primer
ccaccccttgtctcaacact

SEQ ID NO: 24 - DNA, 20 bp, Artificial Sequence
  FOXI3 reverse primer
ttgctcagttgcaaggtgtc

SEQ ID NO: 25 - DNA, 23 bp, Artificial Sequence
  GSC forward primer
gaggagaaagtggaggtctggtt

SEQ ID NO: 26 - DNA, 21 bp, Artificial Sequence
  GSC reverse primer
ctctgatgaggaccgcttctg

SEQ ID NO: 27 - DNA, 20 bp, Artificial Sequence
  KDR forward primer
tttttgcccttgttctgtcc

SEQ ID NO: 28 - DNA, 20 bp, Artificial Sequence
  KDR reverse primer
tcattgttcccagcatttca

SEQ ID NO: 29 - DNA, 20 bp, Artificial Sequence
  LHX1 forward primer
atcctggaccgctttctctt

SEQ ID NO: 30 - DNA, 20 bp, Artificial Sequence
  LHX1 reverse primer
gtaccgaaacaccggaagaa

SEQ ID NO: 31 - DNA, 20 bp, Artificial Sequence
  MEGALIN forward primer
tgtgatgcagccatcgaact

SEQ ID NO: 32 - DNA, 20 bp, Artificial Sequence
  MEGALIN reverse primer
tgcatttggggaggtcagtc

SEQ ID NO: 33 - DNA, 20 bp, Artificial Sequence
  MEOX1 forward primer
aaagtgtcccctgcattctg

SEQ ID NO: 34 - DNA, 20 bp, Artificial Sequence
  MEOX1 reverse primer
cactccagggttccacatct

SEQ ID NO: 35 - DNA, 21 bp, Artificial Sequence
  MIXL1 foward primer
acgtctttcagcgccgaacag

SEQ ID NO: 36 - DNA, 21 bp, Artificial Sequence
  MIXL1 reverse primer
ttggttcgggcaggcagttca

SEQ ID NO: 37 - DNA, 20 bp, Artificial Sequence
  NANOG foward primer
tgcttattcaggacagccct

SEQ ID NO: 38 - DNA, 22 bp, Artificial Sequence
  NANOG reverse primer
tctggtcttctgtttcttgact

SEQ ID NO: 39 - DNA, 19 bp, Artificial Sequence
  OCT4 forward primer
cagtgcccgaaacccacac

SEQ ID NO: 40 - DNA, 20 bp, Artificial Sequence
  OCT4 reverse primer
ggagacccagcagcctcaaa

SEQ ID NO: 41 - DNA, 20 bp, Artificial Sequence
  OSR1 forward primer
ccttccttcaggcagtgaac

SEQ ID NO: 42 - DNA, 20 bp, Artificial Sequence
  OSR1 reverse primer
cggcactttggagaaagaag

SEQ ID NO: 43 - DNA, 20 bp, Artificial Sequence
  OTX2 forward primer
gcagaggtcctatcccatga

SEQ ID NO: 44 - DNA, 20 bp, Artificial Sequence
  OTX2 reverse primer
ctgggtggaaagagaagctg

SEQ ID NO: 45 - DNA, 20 bp, Artificial Sequence
  PAX2 forward primer
caaagttcagcagcctttcc

SEQ ID NO: 46 - DNA, 20 bp, Artificial Sequence
  PAX2 reverse primer
ccacaccactctgggaatct

SEQ ID NO: 47 - DNA, 28 bp, Artificial Sequence
  PAX6 forward primer
acccattatccagatgtgtttgcccgag

SEQ ID NO: 48 - DNA, 26 bp, Artificial Sequence
  PAX6 reverse primer
atggtgaagctgggcataggcggcag

SEQ ID NO: 49 - DNA, 20 bp, Artificial Sequence
  PAX8 forward primer
gcaaccattcaacctcccta

SEQ ID NO: 50 - DNA, 20 bp, Artificial Sequence
  PAX8 reverse primer
ctgctgctgctctgtgagtc

SEQ ID NO: 51 - DNA, 20 bp, Artificial Sequence
  SIX2 forward primer
ctggagagccaccagttctc

SEQ ID NO: 52 - DNA, 20 bp, Artificial Sequence
  SIX2 reverse primer
gctgcgactcttttccttga

SEQ ID NO: 53 - DNA, 20 bp, Artificial Sequence
  SIX4 forward primer
cccaagatggagggtctgta

SEQ ID NO: 54 - DNA, 20 bp, Artificial Sequence
  SIX4 reverse primer
tgtgcttccatctgaagtgc

SEQ ID NO: 55 - DNA, 20 bp, Artificial Sequence
  SOX1 forward primer
caatgcggggaggagaagtc

SEQ ID NO: 56 - DNA, 20 bp, Artificial Sequence
  SOX1 reverse primer
ctctggaccaaactgtggcg

SEQ ID NO: 57 - DNA, 20 bp, Artificial Sequence
  SOX3 forward primer
agaccaggaccgtgtgaaac

SEQ ID NO: 58 - DNA, 20 bp, Artificial Sequence
  SOX3 reverse primer
gtcgatgaatggtcgcttct

SEQ ID NO: 59 - DNA, 19 bp, Artificial Sequence
  SNAI1 forward primer
cccacatccttctcactgc

SEQ ID NO: 60 - DNA, 20 bp, Artificial Sequence
  SNAI1 reverse primer
gtcagcctttgtcctgtagc

SEQ ID NO: 61 - DNA, 20 bp, Artificial Sequence
  SOX17 forward primer
cgcacggaatttgaacagta

SEQ ID NO: 62 - DNA, 20 bp, Artificial Sequence
  SOX17 reverse primer
ggatcagggacctgtcacac

SEQ ID NO: 63 - DNA, 20 bp, Artificial Sequence
  TBX6 forward primer
aagtaccaaccccgcataca

SEQ ID NO: 64 - DNA, 20 bp, Artificial Sequence
  TBX6 reverse primer
taggctgtcacggagatgaa

SEQ ID NO: 65 - DNA, 20 bp, Artificial Sequence
  UMOD forward primer
aaacccatgccacttacagc

SEQ ID NO: 66 - DNA, 20 bp, Artificial Sequence
  UMOD reverse primer
cggtcttcaggctgactttc

SEQ ID NO: 67 - DNA, 20 bp, Artificial Sequence
  WT1 forward primer
gggtacgagagcgataacca

SEQ ID NO: 68 - DNA, 20 bp, Artificial Sequence
  WT1 reverse primer
tctcaccagtgtgcttcctg